(19) United States
(12) Patent Application Publication
(10) Pub. No.: US 2026/0007839 A1
(43) Pub. Date: Jan. 8, 2026

(54) TUNED LOOP TO IDENTIFY VEIN AND BLOOD VESSELS

(71) Applicant: Southern Methodist University, Dallas, TX (US)

(72) Inventors: Jungchih Chiao, Grand Prairie, TX (US); Sen Bing, Dallas, TX (US); Mao-Hsiang Huang, Irvine, CA (US)

(21) Appl. No.: 19/257,841

(22) Filed: Jul. 2, 2025

Related U.S. Application Data
(60) Provisional application No. 63/668,416, filed on Jul. 8, 2024.

(51) Int. Cl. A61M 5/42; G16H 30/40; G16H 40/63

(52) U.S. Cl. CPC A61M 5/427; G16H 30/40; G16H 40/63

(57) ABSTRACT

Provided herein are systems and methods for detecting blood vessels, including a system including a resonator mounted on a substrate; and a processor connected to the resonator; wherein the resonator and the processor are configured to detect at least one of resonant frequencies and reflection coefficient magnitudes when the resonator is applied to a skin surface of a subject.

### CROSS-REFERENCE TO RELATED APPLICATIONS

This application claims priority to U.S. Provisional Application Ser. No. 63/668,416, filed Jul. 8, 2024, the entire contents of which is incorporated herein by reference.

### STATEMENT OF FEDERALLY FUNDED RESEARCH

This invention was made with government support under 1929953 awarded by the National Science Foundation. The government has certain rights in the invention.

### TECHNICAL FIELD

The present invention relates in general to blood vessel detection, and more particularly, to the use of resonators to detect blood vessels.

### BACKGROUND

Without limiting the scope of the disclosure, its background is described in connection with the use of a tuned loop resonator to detect blood vessels.

Venipuncture is the most common medical procedure applied to obtain blood samples from a vein for clinical investigations [1]. Venipuncture is routinely used for blood donation and blood transfusion to patients [1]. The procedure is invasive and depends on individual phlebotomists' manual operation. Although venipuncture has been a routine procedure in large populations, with an estimated 1.4 billion cases a year in the US reported by the Center for Health Statistics, NHAMCS, in 2016, errors frequently occur. The 2021 National Hospital Ambulatory Care Survey reports that over a quarter (41.8 million) of all emergency department (ED) visits in the US involve the placement of an intravenous (IV) catheter for parenteral fluid administration [2]. Rapidly establishing an IV catheter poses a challenge, especially in patients with difficult venous access (DVA), characterized by a lack of readily visible or palpable veins [3]. Difficult venous access is typically defined as experiencing at least two failed IV attempts [4]. Particularly, first-time intravenous insertion success rates range from 53% to 86% [5]-[15] in the pediatric and adult populations. Initial success rates in infants may be even lower at 33% [14], [15]. If failed on the first attempt, the phlebotomist sometimes needs between 2 and 10 attempts to complete the procedure successfully. Reducing the number of venipuncture attempts is a priority in healthcare, given the distressing and painful nature of multiple attempts, such as fainting caused by vasovagal syncope [16], and the associated costs to healthcare [17].

Specific and individual patient factors contribute to insertion failure. Obesity may contribute to challenges in peripheral venous (PV) access, attributed to pathophysiologic changes associated with excess weight. The accumulation of fat in the subcutaneous tissues may result in the presence of deeply located peripheral veins, posing difficulties in catheterization. This issue of PV access difficulty has been observed in morbidly obese patients in various settings, including in operating rooms and emergency conditions [18]-[20]. Age complicates the venipuncture due to the thin and loose skin as well as aging blood vessels [21]. Other factors [4], [6], [10]-[13], [22], [23], such as skin colors, diabetes, intravenous drug use, chemotherapy, chronic medical conditions, and needle phobia, could aggravate the difficult venous access. Despite the factors from patients, the shortage of phlebotomists further exacerbates the issue [24], [25]. Sonmez et al. reported that only 38% of 1347 tubes of blood samples were collected by 73 nurses under the safety mechanism [26]. A variety of adverse complications are encountered due to improper site selection and excessive venipuncture caused by the DVA complexity. Hematoma formation is the most common complication of venipuncture [27]. Excessive probing to find the vein is the main reason that causes blood to leak into the tissues, resulting in a bruise. Moreover, it creates fear and phobia in patients with repeated failed venipunctures. Serious cases, including nerve injury, mistaken arterial punctures, thrombus, and syncope, have been reported due to the close distance between the nerves, arterial, and veins [28], [29].

Besides human judgment with the caregiver's visual inspection or touch to palpate the vein with fingers, which is the typical means, several technological approaches have been reported to improve venipuncture success rates [30]. Ultrasound-guided methods are used for locating veins in some reports [22], [31]-[33]. However, ultrasound images are noisy, blurry, and sensitive to placement. It requires the vein to be relatively large [34]. Additionally, ultra-sound requires expensive equipment and additional trained expertise to obtain meaningful images while simultaneously cannulating the vein [35], making it not universally available. Visible-light transillumination has been utilized to enhance the visualization of veins [36]. Its application has been extended to the varicose vein treatment, as reported in [37]. However, this method necessitates a darkened room, causing inconvenience, and its results have shown inconsistency with instances of burns due to the high intensity of required light [35]. Infrared (IR) imaging emerges as a potential solution due to its deeper penetration into human tissues compared to visible light [38]. Additionally, veins containing de-oxygenated hemoglobin-rich blood absorb light significantly at the near-infrared (NIR) wavelengths (740 760 nm) over several centimeters in the tissues. This property is exploited by NIR spectroscopy and can effectively distinguish veins from surrounding tissue [39]. Several commercial devices, such as VEINVIEWER® [40], ACCUVEIN® [41], VEINSITER [23], and VASCULUMINATOR®[42], employ NIR for vascular access procedures. Despite their valuable clinical functions, these NIR devices face limited adoption in clinics due to their high costs, bulky instruments, and user-machine interface. NIR is limited to about a 3-mm depth in tissues, which may not be deep enough as the mean value is 3.1 mm for the basilic veins [43]. These factors prevent them from practical and wide uses as studies have shown no significant benefit by using them compared to manual procedures [44], [45]. Besides, the costs limit their use in economically disadvantaged areas. It is critically important to find a means to locate veins noninvasively, economically, and accurately.

### SUMMARY

As embodied and broadly described herein, an aspect of the present disclosure relates to a system for detecting a blood vessel, comprising: a resonator mounted on a substrate; and a processor connected to the resonator; wherein the resonator and the processor are configured to detect at least one of resonant frequencies and reflection coefficient magnitudes when the resonator is applied to a skin surface of a subject, which can be a human subject or patient, or an animal. In one aspect, the resonator is a tuned loop resonator comprising an outer loop and a metal pad disposed within the outer loop and concentric with the outer loop such that a gap width between the outer loop and the metal pad is constant. In another aspect, the metal pad has a hole at a center thereof, the hole configured to receive an injection needle or visualization of the skin. In another aspect, the processor is a network analyzer, a vector network analyzer, or a spectrum analyzer. In another aspect, the resonator and the processor are configured to detect differences in the at least one of the resonant frequencies and the reflection coefficient magnitudes when the resonator is applied to more than one location on the skin surface. In another aspect, the resonator and the processor are configured to detect differences in the at least one of the resonant frequencies and the reflection coefficient magnitudes when the resonator is applied to the skin surface over the blood vessel and when the resonator is applied to the skin surface over tissue surrounding the blood vessel. In another aspect, the resonator and the processor are configured to detect changes in the at least one of the resonant frequencies and the reflection coefficient magnitudes over time when the resonator is applied to the skin surface. In another aspect, the resonator is applied to the skin surface over a blood vessel. In another aspect, the system further comprises one or more additional resonators configurable in an array on the skin surface. In another aspect, the resonator is selected from RF, microwave, millimeter-wave, and terahertz-frequency sensors; infrared, near-infrared, and ultraviolet optical sensors; magnetic sensors; acoustic sensors; and combination thereof.

As embodied and broadly described herein, an aspect of the present disclosure relates to a kit for a system for detecting blood vessels, comprising: a resonator mounted on a substrate; and a processor connected to the resonator; wherein the resonator and the processor are configured to detect at least one of resonant frequencies and reflection coefficient magnitudes when the resonator is applied to a skin surface of a subject, which can be a human subject or patient, or an animal; and one or more tools for manipulation of the resonator and instructions for use of the kit. In one aspect, the resonator is a tuned loop resonator comprising an outer loop and a metal pad disposed within the outer loop and concentric with the outer loop such that a gap width between the outer loop and the metal pad is constant. In another aspect, the metal pad has a hole at a center thereof, the hole configured to receive an injection needle or visualization of the skin. In another aspect, the processor is a network analyzer, a vector network analyzer, or a spectrum analyzer. In another aspect, the resonator and the processor are configured to detect differences in the at least one of the resonant frequencies and the reflection coefficient magnitudes when the resonator is applied to more than one location on the skin surface. In another aspect, the resonator and the processor are configured to detect differences in the at least one of the resonant frequencies and the reflection coefficient magnitudes when the resonator is applied to the skin surface over the blood vessel and when the resonator is applied to the skin surface over tissue surrounding the blood vessel. In another aspect, the resonator and the processor are configured to detect changes in the at least one of the resonant frequencies and the reflection coefficient magnitudes over time when the resonator is applied to the skin surface. In another aspect, the resonator is applied to the skin surface over a blood vessel. In another aspect, the system further comprises one or more additional resonators configurable in an array on the skin surface. In another aspect, the resonator is selected from RF, microwave, millimeter-wave, and terahertz-frequency sensors; infrared, near-infrared, and ultraviolet optical sensors; magnetic sensors; acoustic sensors; and combination thereof.

As embodied and broadly described herein, an aspect of the present disclosure relates to a method of detecting a blood vessel, comprising: providing a subject, which can be a human subject or patient, or an animal, in need of detection of the blood vessel; providing a system for detecting the blood vessel comprising: a resonator mounted on a substrate; and a processor connected to the resonator; wherein the resonator and the processor are configured to detect at least one of resonant frequencies and reflection coefficient magnitudes when the resonator is applied to a skin surface of the subject; applying the resonator to a plurality of locations on a skin surface of the subject; detecting the at least one of the resonant frequencies and the reflection coefficient magnitudes at each of the plurality of locations on the skin surface; and identifying the blood vessel from differences among the at least one of the resonant frequencies and the reflection coefficient magnitudes detected. In one aspect, the resonator is a tuned loop resonator comprising an outer loop and a metal pad disposed within the outer loop and concentric with the outer loop such that a gap width between the outer loop and the metal pad is constant. In another aspect, the metal pad has a hole at a center thereof, the hole configured to receive an injection needle or visualization of the skin. In another aspect, the processor uses a network analyzer, a vector network analyzer, or a spectrum analyzer. In another aspect, the resonator and the processor are configured to detect differences in the at least one of the resonant frequencies and the reflection coefficient magnitudes when the resonator is applied to more than one location on the skin surface. In another aspect, the resonator and the processor are configured to detect differences in the at least one of the resonant frequencies and the reflection coefficient magnitudes when the resonator is applied to the skin surface over the blood vessel and when the resonator is applied to the skin surface over tissue surrounding the blood vessel. In another aspect, the resonator and the processor are configured to detect changes in the at least one of the resonant frequencies and the reflection coefficient magnitudes over time when the resonator is applied to the skin surface. In another aspect, the resonator is applied to the skin surface over a blood vessel. In another aspect, the system further comprises one or more additional resonators configurable in an array on the skin surface. In another aspect, the resonator is selected from RF, microwave, millimeter-wave, and terahertz-frequency sensors; infrared, near-infrared, and ultraviolet optical sensors; magnetic sensors; acoustic sensors; and combination thereof.

As embodied and broadly described herein, an aspect of the present disclosure relates to a processor-implemented method for detecting a blood vessel and blood flow in the blood vessel using a machine learning model, wherein the method comprises: performing, using a processor, a scan of a tissue with a resonator sensor to conduct one or more raster scans that generate pixels of data to create an image; wherein the resonator sensor detects a reflection coefficient s₁₁ within a range of frequencies, wherein a magnitude and phase of su as a function of frequency are recorded; and determining the resonant frequency and the magnitude at a location on the skin to generate a first and a second images, wherein a first image contains pixels of the resonant frequencies and the second image contains pixels of the |s₁₁|, wherein the resonant-frequency image shows a location of a vein as a line of pixels wherein the resonant frequencies of the vein under the skin are lower than neighbor pixels. In one aspect, a frequency with a minimum |s₁₁| is treated as a resonant frequency. In another aspect, the method further comprises combining a data set of the first and second images to generate a resonant-frequency map of the vein location by: normalizing a datasets of the first and second images to a minimum and maximum range, wherein the normalization ranges between 0 and 1, with 0 being a lowest resonant frequency and 1 being a highest resonant frequency in the resonant-frequency map, and 0 being the lowest |s₁₁| and 1 being the highest |s₁₁| in the |s₁₁|map, respectively; generating a first and a second map with the scale from 0 to 1, wherein a weighting value w is used to emphasize the influence of a specific map, wherein the value w is between 0.01 and 0.99; and wherein the first map is given a value of x while the second map is given 1-w; generating an integrated map with each pixel containing a sum of a pixel value times w from the first map and the pixel value times (1-w) from the second map; wherein, if the resonant-frequency map has a line of pixels with values lower than others, the resonant-frequency map is given a larger value w; or wherein, if the |s₁₁| map has a line of pixels with values lower than others, the |s₁₁|map is given a larger value w, while the resonant-frequency map is given a smaller value of (1-w); generating a series of integrated maps comprising a series of varying w values; comparing a contrasts of the first and second, wherein a highest contrast map gives the clearer vein location, as a line, and determines the optimal w value; and using an |s₁₁| image to find a vein location. In another aspect, the method further comprises obtaining an ultrasound image of the tissue with an ultrasound transducer array. In another aspect, the resonator sensor identifies the vein along a medial/lateral and proximal/distal location, and concurrently an ultrasound cross-sectional image indicates the vein depth under the skin. In another aspect, a machine learning model increases a sensitivity of the vein localization by taking two or more scans of the same location of a tissue wherein two or more pixel maps are collected for known vein locations, segmenting the two or more pixel maps into two or more smaller pixel maps for training; rotating the two or more smaller pixel maps to change vein locations; determining a ground truth for each of the two or more smaller pixel maps that contain vein locations in certain pixels or without vein pixels. In another aspect, the method further comprises using the machine learning model on new scans to generate vein locations maps at one or more different depths, wherein optionally a depth of the vein can be verified by an ultrasound transducer.

As embodied and broadly described herein, an aspect of the present disclosure relates to one or more non-transitory computer-readable storage mediums storing one or sequences of instructions, which when executed by one or more processors, causes a method for detecting a blood vessel and blood flow in the blood vessel using a machine learning model, wherein the method comprises: performing, using a processor, a scan of a tissue with a resonator sensor to conduct one or more raster scans that generate pixels of data to create an image; wherein the resonator sensor detects a reflection coefficient su within a range of frequencies, wherein a magnitude and phase of su as a function of frequency are recorded; and determining the resonant frequency and the magnitude at a location on the skin to generate a first and a second images, wherein a first image contains pixels of the resonant frequencies and the second image contains pixels of the |s₁₁|, wherein the resonant-frequency image shows a location of a vein as a line of pixels wherein the resonant frequencies of the vein under the skin are lower than neighbor pixels. In one aspect, a frequency with a minimum |s₁₁| is treated as a resonant frequency. In another aspect, the method further comprises combining a data set of the first and second images to generate a resonant-frequency map of the vein location by: normalizing a datasets of the first and second images to a minimum and maximum range, wherein the normalization ranges between 0 and 1, with 0 being a lowest resonant frequency and 1 being a highest resonant frequency in the resonant-frequency map, and 0 being the lowest |s₁₁| and 1 being the highest |s₁₁| in the |s₁₁|map, respectively; generating a first and a second map with the scale from 0 to 1, wherein a weighting value w is used to emphasize the influence of a specific map, wherein the value w is between 0.01 and 0.99; and wherein the first map is given a value of x while the second map is given 1-w; generating an integrated map with each pixel containing a sum of a pixel value times w from the first map and the pixel value times (1-w) from the second map; wherein, if the resonant-frequency map has a line of pixels with values lower than others, the resonant-frequency map is given a larger value w; or wherein, if the |s₁₁| map has a line of pixels with values lower than others, the |s₁₁|map is given a larger value w, while the resonant-frequency map is given a smaller value of (1-w); generating a series of integrated maps comprising a series of varying w values; comparing a contrasts of the first and second, wherein a highest contrast map gives the clearer vein location, as a line, and determines the optimal w value; and using an |s₁₁| image to find a vein location. In another aspect, the method further comprises obtaining an ultrasound image of the tissue with an ultrasound transducer array. In another aspect, the resonator sensor identifies the vein along a medial/lateral and proximal/distal location, and concurrently an ultrasound cross-sectional image indicates the vein depth under the skin. In another aspect, a machine learning model increases a sensitivity of the vein localization by taking two or more scans of the same location of a tissue wherein two or more pixel maps are collected for known vein locations, segmenting the two or more pixel maps into two or more smaller pixel maps for training; rotating the two or more smaller pixel maps to change vein locations; determining a ground truth for each of the two or more smaller pixel maps that contain vein locations in certain pixels or without vein pixels. In another aspect, the method further comprises using the machine learning model on new scans to generate vein locations maps at one or more different depths, wherein optionally a depth of the vein can be verified by an ultrasound transducer.

### BRIEF DESCRIPTION OF THE DRAWINGS

For a more complete understanding of the features and advantages of the present invention, reference is now made to the detailed description of the disclosure along with the accompanying figures, in which:

FIG. 1A shows a configuration of the tuned sensor with radius b=4.7 mm, loop width w =0.9 mm, stub length L=1.5 mm, and tuning gap d=0.2 mm. FIG. 1B shows a photograph of the tuned sensor on a FR4 substrate.

FIG. 2 shows a simulation setup in which the vein structure consists of a vessel wall and blood.

FIG. 3A shows a comparison of documented dielectric constants of skin, vessel wall, and blood [57]. FIG. 3B shows a comparison of documented conductivities of skin, vessel wall, and blood [57].

FIG. 4 shows a comparison of reflection coefficients for skin with and without a vein underneath.

FIGS. 5A and 5B show cross-sections of electric field distributions from the tuned loop resonator. FIG. 5A shows a top view, and FIG. 5B shows a side view.

FIG. 6 shows a comparison of simulated resonant frequencies with a vein in the depth from 1.7 mm to 6 mm, with a step depth size of 0.3 mm.

FIG. 7A shows a photo of the sensor applied onto the skin in measurements. FIG. 7B shows a setup of measurement. FIG. 7C shows a photo of the positioning system for measurement stabilization.

FIGS. 8A-8D show measured locations for skin and skin with veins underneath in the male subject's forearms with the reference of near-infrared (NIR) imager. FIG. 8A shows a left forearm without a vein underneath in the pen-circled area #1. FIG. 8B shows a left forearm with a vein underneath in the pen-circled area #3. FIG. 8C shows a right forearm without a vein underneath. FIG. 8D shows a right forearm with a vein underneath.

FIG. 9 shows a comparison of reflection coefficients in measurements for the skin with and without a vein underneath.

FIGS. 10A and 10B show comparisons of measured resonant frequencies for the male subject with and without a vein underneath. Measurements were repeated five times in each location. FIG. 10A shows a comparison for the left arm, and FIG. 10B shows a comparison for the right arm.

FIGS. 11A-11D show measured locations for the skin with and without a vein in the female subject's forearms with the reference of the NIR imager. FIG. 11A shows measured locations for the left forearm without a vein underneath. FIG. 11B shows a measured location for the left forearm with a vein underneath. FIG. 11C shows measured locations for the right forearm without a vein underneath. FIG. 11D shows measured locations for the right forearm with a vein.

FIGS. 12A and 12B show comparisons of measured resonant frequencies for the female subject with and without a vein underneath. Measurements were repeated five times in each location on the left forearm (FIG. 12A) and the right forearm (FIG. 12B).

FIG. 13 a comparison of measured resonant frequencies in various locations on the skin of the forearm, including the distal, middle, and upper regions.

FIG. 14 shows measured locations for arteries in the male and female subjects with the NIR image references.

FIGS. 15A and 15B show comparisons of measured resonant frequencies for the skin, vein, and artery. Measurements were repeated five times in each measured location on the male subject (FIG. 15A) and the female subject (FIG. 15B).

FIGS. 16A-16D show cross sections showing SAR field distributions from the tuned loop resonator. FIG. 16A shows a top view without a vein underneath the skin. FIG. 16B shows a side view without a vein underneath the skin. FIG. 16C shows a top view with a vein underneath the skin. FIG. 16D shows a side view with a vein underneath the skin.

FIG. 17 shows a setup of skin dielectric properties measurement using the dielectric probe kit.

FIG. 18A shows a comparison of measured dielectric constants of skin with and without a vein underneath. FIG. 18B shows a comparison of measured conductivity of skin with and without a vein underneath.

FIG. 19 shows an embodiment of the present invention in which the sensor's metal pad has a hole in its center to receive an injection needle, a needle holder to receive the injection needle, and a visual indication indicating when the sensor is centered over a blood vessel.

FIG. 20 shows a flowchart for a method embodiment of the present invention.

FIG. 21A shows a configuration of the tuned sensor with a radius b=5.2 mm, a loop width w=0.7 mm, and a tuning gap d=1.5 mm. FIG. 21B shows a simulation model in which the vein structure consists of blood inside vessel wall embedded within the skin.

FIG. 22 shows a comparison of reflection coefficients for skin with and without a vein underneath.

FIGS. 23A and 23B show cross sections of electric field distributions from the tuned loop resonator: top view (FIG. 23A) and side view (FIG. 23B).

FIGS. 24A and 24B show resonant frequencies by a raster scan across the blood vessel, located at the zero, under the skin: MCV (FIG. 24A) and CV (FIG. 24B).

FIGS. 25A and 25B show images generated from the raster scan for a vein at a depth of 3 mm. The vein is located at (X=0): resonant frequencies (FIG. 25A) and |s₁₁| (FIG. 25B).

FIG. 26 shows a comparison of reflection coefficients with Y varied from −10 mm to +10 mm.

FIG. 27 shows a confusion matrix that illustrates the performance of a decision tree classification model when applied to the experimental dataset. The model achieved 82.09% overall accuracy in distinguishing between the presence (1) and absence (0) of veins beneath the microwave loop resonator sensor.

FIG. 28 shows an example of a machine learning technique to train contrast recognition.

FIG. 29 shows another example of a machine learning technique to train contrast recognition.

FIGS. 30A and 30B show configurations of the resonator sensor integrated with one or more ultrasound transducers.

FIG. 31 shows a two-resonator configuration for such a differential comparison.

### DETAILED DESCRIPTION OF THE INVENTION

Illustrative embodiments of the system of the present application are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms such as “above,” “below,” “upper,” “lower,” or other like terms to describe a spatial relationship between various components or to describe the spatial orientation of aspects of such components should be understood to describe a relative relationship between the components or a spatial orientation of aspects of such components, respectively, as the device described herein may be oriented in any desired direction.

The present inventors recognized that low-power microwave sensing with nonionizing radiation can be used for vein locating. Nonionizing radiation with a low power density does not present a potential risk of damaging tissues [46]. The dielectric properties of blood vessels are significantly different from those of skin and muscles because of the high water content in the blood plasma. Due to the high contrast in dielectric properties between tissues and vessels, the interactions of electromagnetic fields with different tissues affect wave scattering and provide noninvasive real-time sensing. The presence of veins under the skin can be detected by evaluating the resonant frequency shifts and magnitude changes of reflection coefficients in measurements. However, while the present invention is discussed herein in terms of low-power microwave sensing, aspects of the present invention include other types of sensors, including these non-limiting examples: RF, millimeter-wave, and terahertz-frequency sensors; infrared, near-infrared, and ultraviolet optical sensors; magnetic sensors; acoustic sensors; and combinations thereof.

Recent RF devices and integrated circuits are cost-effective with low power consumption. Their sizes can be small and integrated into a wearable device form factor for clinical uses. The advances in electronics make an affordable microwave sensing wearable possible. This disclosure demonstrates an efficient microwave sensing element that can be conformed to the human forearm to detect the presence of veins under the skin.

The present inventors show that it is possible to have linear arrays of sensors that allow detection of fluid vessels such as blood vessels (veins and/or arteries), ducts (e.g., bile ducts, urethra, cerebrospinal fluid), that are capable of measuring/sensing water and the lack of water (e.g., in the presence of kidney stones, water/fat deposits such as cysts or tumors), detect and/or measure internal bleeding, changes in fluid or blood pressure, fluid or blood flow, blood clots, etc. It is also possible to use arrays (2 dimensional and/or angled) that can steer the beams to detect deeper into the target tissue. For example, the sensor(s) and/or arrays can be placed in a wearable device, such as a sock or sleeve to measure changes in fluid flow, e.g., loss of blood flow in the extremities of diabetic patients. It is also possible to use the sensor(s) and/or arrays to provide an image for venipuncture in which the array allows the phlebotomist to dial-into the vein and using a sensor that has an opening in the middle, place the sensor over the spot and insert the needle into the vein without the need to remove or lift the sensor. When packaged into a sterile packaging, the sensor can be used as a single-use sensor for venipuncture or insertion into any vessel, e.g., epidurals, injections, or even biopsies.

### Sensor Design and Simulations.

Compared to waveguides, planar resonators usually have smaller sizes [47], which will be beneficial for the use in vein finding, particularly on children's arms. Resonating cavities [48]-[50] are not suitable for comfortably interfacing on the human forearm. Their measurements are limited for media with high dielectric losses [51]. Split ring resonators (SRRs) have found sensing applications in planar circuits [52]-[55]. The energy coupling from the outer ring to the inner ring provides the desired resonance. However, the coupling introduces insertion losses, and the two rings bring multiple harmonics, which reduce the quality factor of the resonance of interest. Additionally, both rings are susceptible to environmental changes, particularly concerning wearables. Related to SRR, the complementary split-ring resonator (CSRR) has also been used for microwave sensing [56]. However, CSRRs exhibit higher insertion losses because the inner split ring, responsible for the required resonance, is indirectly excited by the current in the outer ring. This indirect excitation results in a reduced penetration depth of the electric fields into the tissues.

Planar loop resonators seem to be more appropriate, with the advantages of being compact and wearable. However, with the impedance matching issue [46], the poor resonance at the microwave frequencies results in insufficient penetration of fields into tissues. While employing a dynamic matching circuit can achieve a high-quality factor, it results in bulkiness and design limitations. The technical challenge lies in attaining a high-quality factor without compromising the features of being planar and with a small footprint. The inventors previous work [58] showed the development of a self-tuned method for impedance matching in a planar loop resonator by embedding a concentric metal pad, keeping the resonator device architecture simple but efficient. The presence of a center pad provides additional distributed capacitances from the gap between the loop and the metal pad and additional mutual inductance between metal strips across the gap owing to coupled magnetic fields. The gap spacing between the loop and pad tunes the distributed reactance and matches the port impedance at the desired operating frequency. Without changing the overall loop size or adding additional tuning circuits outside of the loop, the resonance of the planar loop can be improved significantly. The loop resonators have been made into compact forms with high resonance performance for near-field sensing. Applications have been investigated for human hydration monitoring [61]-[64], subcutaneous implant localization [65], [66], breast cancer detection or imaging [67], [68], and skin cancer distinction [69]. The high-quality factors also enhanced transcutaneous wireless power transfer [70]. In agriculture, the tuned resonator can be used to detect water content percentages of fruits or vegetables [71].

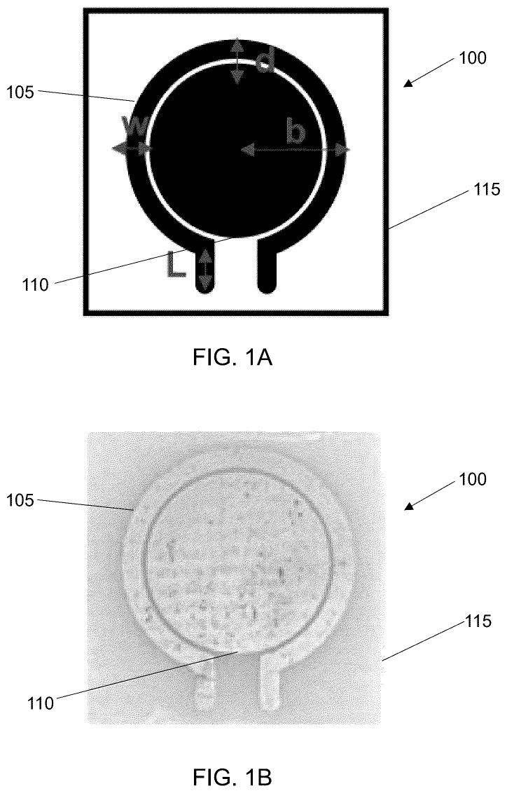

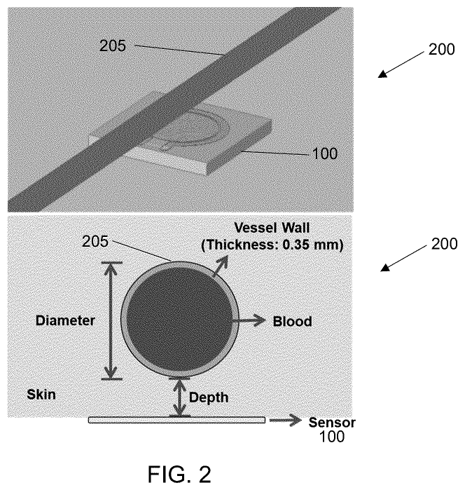

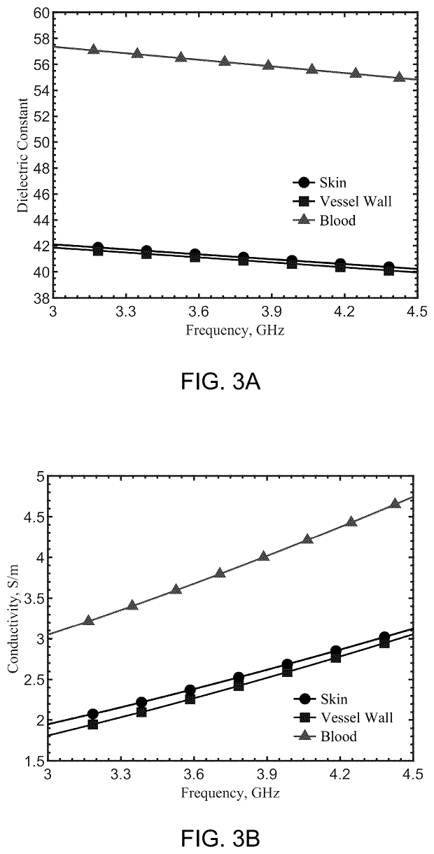

This disclosure utilizes a similar tuned loop resonator structure to find the vein under the skin. A rigid substrate capable of withstanding pressure ensures consistent and firm contact with the skin and enhances measurement accuracy. The tuned loop resonator (also referred to herein as “sensor”) in this study was designed and fabricated on a rigid single-layer FR4 substrate with a dielectric constant of 4.4 and a thickness of 1.5 mm. The same parameters were used in simulations. The tuned loop resonator 100 consisted of a loop 105 as the resonating element and a metal pad 110 as the tuning element on the substrate 115, as shown in FIG. 1A. The copper pattern was etched after photolithography was applied. A photo of the loop resonator 100 is shown in FIG. 1B, including the loop 105, the metal pad 110, and the substrate 115. (Herein, all references to the sensor or to the tuned loop resonator indicate embodiments of the sensor as described herein, including the sensor 100.) The radius b of the loop resonator 100 was 4.7 mm with a connecting stub length L=1.5 mm, which was prepared for a connector to the measurement port. The metal width w was 0.9 mm. The gap d between the loop 105 and the center metal pad 110 was chosen to be 0.2 mm, which tuned the resonance below −55.27 dB in a simulation model in which the sensor 100 was placed on the skin with a vein underneath. FIG. 2 shows the simulation 200, including a vein 205 and the resonator 100. The simulation model consisted of tissues and a single vein. In humans, superficial veins, including cephalic veins (CV) and median cubital veins (MCV), are commonly used for venipuncture [43]. The vein sizes and depths vary per person. According to [43], [72], the average depth range of the vein is 1.7-3 mm, and the average diameter ranges 1.7-4.9 mm. In this disclosure, the simulated vein depth was set at 2 mm with a diameter of 3.6 mm. The vessel wall in the simulation was assumed to be 0.35 mm, according to [73], [74]. The dielectric properties used for skin, blood, and vessel walls were obtained from a documented library in [57], with plots shown in FIGS. 3A and 3B, showing the dielectric constant and conductivity of the three materials between 3 and 4.5 GHZ. It should be noted that the database is highly generalized, so discrepancies with measurements on individuals are expected.

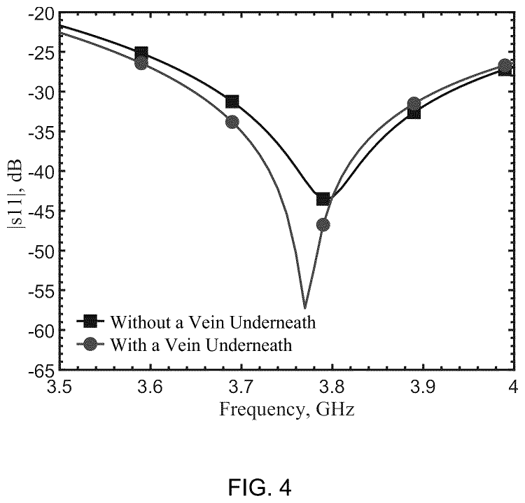

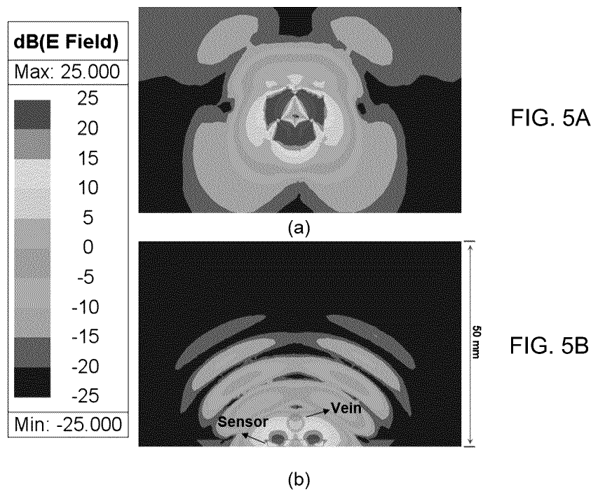

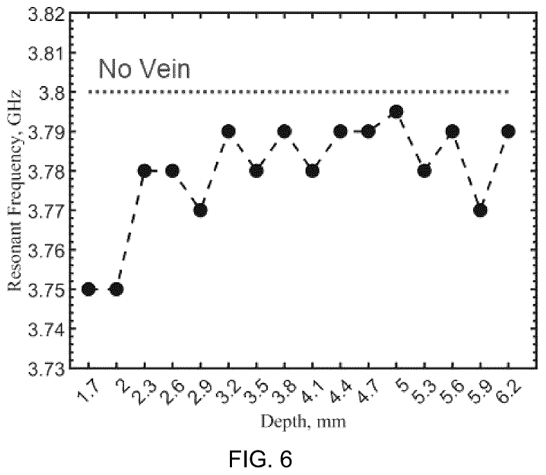

FIG. 4 shows the simulated reflection coefficient comparison with and without the vein underneath utilizing the documented data. With a vein underneath, the resonance is at 3.77 GHZ with s₁₁ of −57.27 dB, whereas it is 3.79 GHz with su of −43.52 dB when there is no vein underneath. The robust resonance provides confined electrical fields deep enough to penetrate through the skin and reach the vein, providing sufficient sensitivity to detect the effective permittivities change contributed by the presence of the vein. FIGS. 5A and 5B show strong fields across the gap and around the loop. The −10-dB attenuation depth from the surface of the loop, in FIG. 5B, is 3 mm, covering the vein depth range [75], and the −20-dB attenuation depth is 13 mm. Considering the deep field into the skin, simulations with a vein in a wider range of depths from 1.5 mm to 6 mm were conducted than that of 1.7-3 mm according to [75]. Resonant frequencies are compared in FIG. 6 showing the vein can be detectable up to a depth of 6 mm. However, the frequency shift becomes smaller after 3 mm due to the near-field sensor having a maximum field concentrated within the depth of 3 mm, matching the field distribution shown in FIG. 5B.

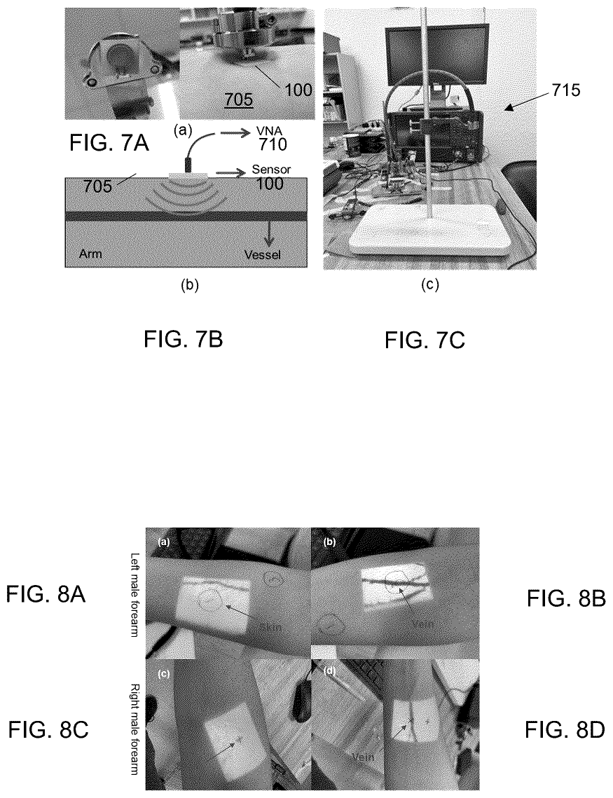

Vein Detection Measurements. Vein Detection. The inside of the forearm (antecubital area of the ante-brachium) is the most common area for venipuncture. The median cubital vein (MCV) and cephalic vein (CV) are the common targets. The antecubital area skin is relatively thin. The sensor 100 is applied with sufficient pressure onto the forearm skin 705 and connected to, e.g., a vector network analyzer (Keysight PNA N5227B) 710, as shown in FIGS. 7A and 7B, respectively (the VNA 710 is not shown in FIG. 7A). Due to the high sensitivity of the tuned sensor, mechanical tension changes on the coaxial cable induce small movements between the arm and cable, causing measurement fluctuations. To improve stability, a laboratory positioning system 715 with bracket mounting is used to stabilize the sensor 100 (not shown), as shown in FIG. 7C. The person can freely move the forearm, allowing the sensor 100 to be placed at different measurement points. The human subjects research protocol ID is SMU-IRB-22-205, approved by the Southern Methodist University IRB committee.

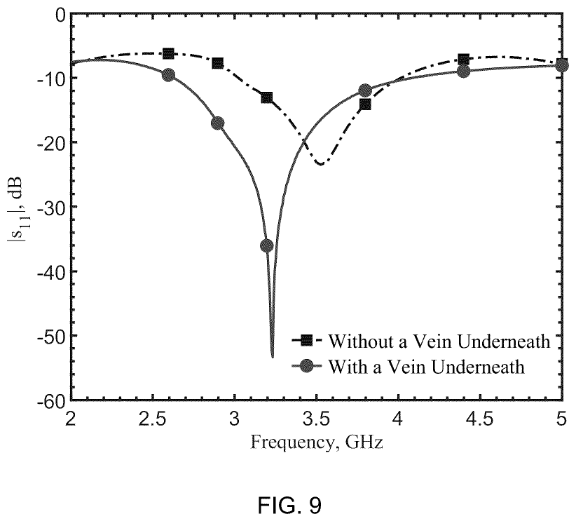

The inventors found that in vivo di-Measurements in this disclosure compared differences in females and males. It was found that female skin is relatively thinner with a lower dielectric constant compared to male skin before 10 GHz. Similarly, Mayrovitz et al. [77], reported that male skin has a higher tissue dielectric constant than female skin. To investigate skin condition differences with respect to gender, a male and a female subject of the same age and with similar body sizes were measured on both left and right arms at selected locations with or without clear veins underneath the skin. A commercial near-infrared (NIR) vein finder system (UMTEC-ZD001, USA) was applied as the reference. Due to the vein locations being different on two forearms and on different persons, the measured physical locations with their distances from the elbows or hands were different. In the male subject's left forearm, two measurement points were selected with the assistance of the NIR finder, with and without a clear vein underneath, as shown in FIGS. 8A and 8B. The circle #1 in the figures on the skin was without a vein and #3 was with a vein. Reflection coefficients are compared in FIG. 9. With a vein underneath, the resonance was 3.232 GHz with |s₁₁| of −53.36 dB, whereas it was 3.527 GHz with su of −23.48 dB when there was no vein underneath. Excellent resonance was expected as the simulation was based on tuning with a vein underneath. The frequency shift was 0.295 GHZ.

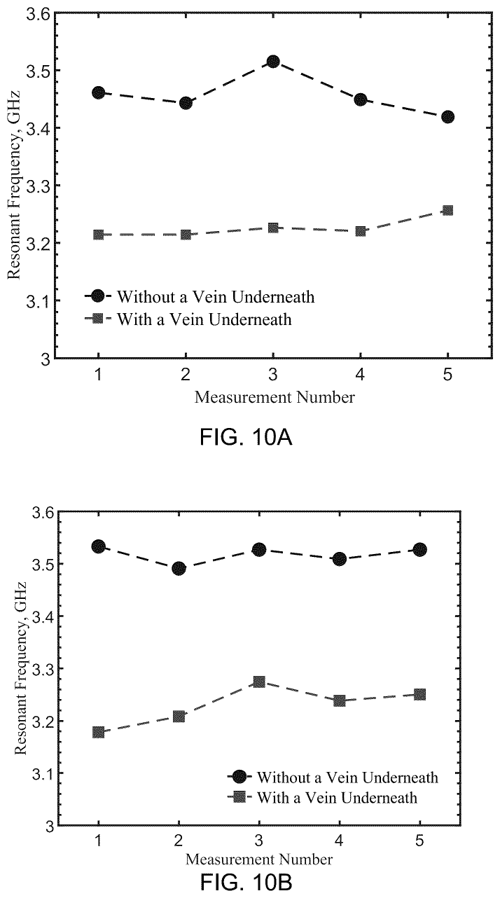

The applied contact pressure onto the sensor and skin may affect the s₁₁. During measurement, some pressure variations may still exist. The pressure and force applied on the skin compress the internal tissues, squeeze and rearrange relative locations of cells, blood vessels, fat, and water, causing the overall structure changes. This can change the effective permittivity and conductivity in the tissues, even if the field distribution from the resonator remains the same. Thus, measurements were repeated and recorded five times in each location. Each time, the sensor was removed and placed again in the same location. A verbal confirmation from the subject verified if the person felt the pressure was similar to that in the previous measurement. The resonant frequencies for individual measurements are compared in FIG. 10A. The measured resonant frequency with the vein underneath was around 3.226 GHZ, averaged from five measurements at 3.214, 3.214, 3.226, 3.220, and 3.256 GHZ, while the average resonant frequency without a vein underneath was 3.457 GHz from five measurements at 3.461, 3.443, 3.515, 3.449 and 3.419 GHz. The variation ranges were 42 and 96 MHz with and without a vein underneath. Similarly, the measurements were conducted on the right forearm of the same male subject, with and without a vein underneath, as shown in FIGS. 8C and 8D. The measured results are compared in FIG. 10B. The average resonant frequency was 3.23 GHz with a vein underneath, averaged from five measurements at 3.178, 3.208, 3.275, 3.238, and 3.251 GHz. Without a vein underneath, it was 3.51 GHz, which was averaged from 3.533, 3.491, 3.527, 3.509, and 3.527 GHz. The variation ranges were 96 and 42 MHz with and without a vein underneath. The results were repeatable and distinguishable between the cases with or without a vein underneath in both male subject's forearms. The frequency variations among five measurements in the same location were expected. The frequency variation had an average value of 69 MHz, which was sufficiently small compared to the frequency shift caused by the presence of a vein underneath. An average 259 MHz frequency shift could be found in both forearms when a vein was underneath compared to only the skin.

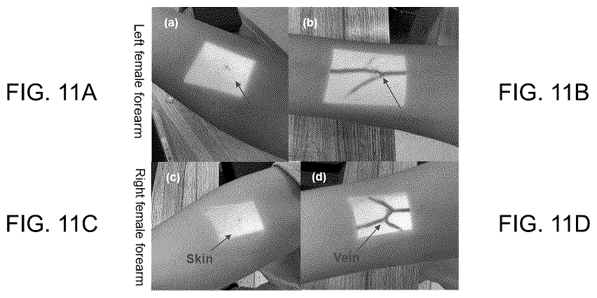

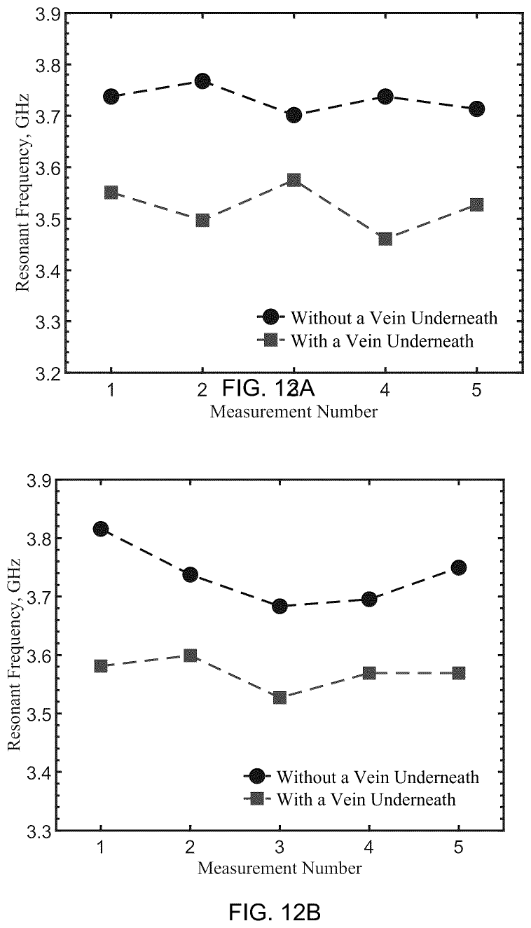

As for the female subject, measurements were accordingly selected on locations similar to the male subject's locations on the left and right forearms, as shown in FIGS. 11A-11D. Measurements were repeated five times in each location for repeatability, and the resonant frequency for each measurement was compared in FIGS. 12A and 12B for the left and right forearms, respectively. The five measurements in the left forearm with a vein underneath were 3.551, 3.497, 3.575, 3.461, and 3.527 GHz, with an averaged resonant frequency of 3.522 GHz. For the case without a vein underneath, the five measured resonant frequencies were 3.737, 3.768, 3.701,3.737, and 3.713 GHz, with an average value of 3.731 GHz. The variation ranges were 114 and 66 MHz with and without a vein underneath. In the right forearm of the same female subject, with a vein underneath, the averaged resonant frequency was 3.569 GHZ from five measurements at 3.581, 3.599, 3.527, 3.569, and 3.569 GHZ, while the one without a vein underneath was 3.736 GHz, which was averaged from 3.816, 3.737, 3.683, 3.695 and 3.749 GHz. The variation ranges were 72 and 13 MHz with and without a vein underneath. The frequency variation was an average value of 96 MHZ in the same location. In the measurements on the female subject, there was an average of 188 MHz shift between the cases with and without a vein underneath.

The distinguishable frequency shift indicated if there was a vein underneath. Additionally, the overall resonant frequency values, including both with and without a vein underneath, were roughly 200 MHz higher compared to those on the male subject. It was expected because the female skin, with less relative thickness, had a lower tissue dielectric constant, as mentioned earlier in [76]-[78], leading to higher resonant frequencies.

### Measured Location Effects.

Under the skin, thicknesses of the epidermis, dermis, and hypodermis layers can vary at different locations on the forearm for an individual [80]. The thickness variations lead to variations of effective dielectric properties, causing changes in the measured resonant frequencies. Additionally, the thicknesses of different types of tissues also affect the pressure effects. For example, the compression on the hypodermis/fat layer may vary the tissue thickness more than the epidermis layer. Thus, the body type with thicker layers of fat may experience a larger range of permittivity change when the skin is pressed by the sensor. Differences in curvatures on the forearm for the same person also mean different pressures may be induced on the skin when the sensor is placed. The potential interference factors from curvature, tissue compression and variations of tissue thickness could affect the measurement results. Thus, further investigations were conducted on different measured locations without a vein underneath along the forearm, including the distal, middle, and upper regions of the male subject. The curvature radius and overall skin thickness increase along the forearm from the distal to upper regions. These measurements can be used for calibration to identify the frequency variations without a vein underneath the forearms.

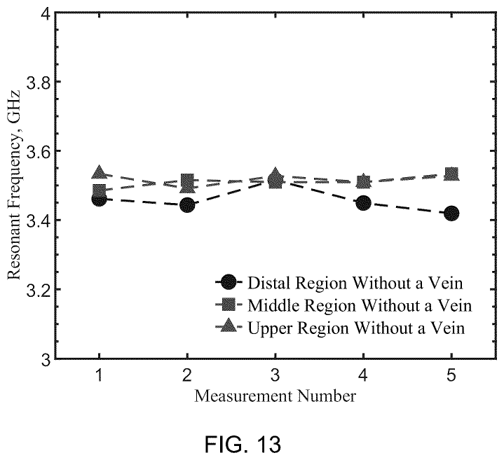

Reflection coefficients for each location were measured five times. FIG. 13 shows the comparison of measured resonant frequencies on the various skin locations. The measured resonant frequencies roughly overlap with the average values of 3.46 GHZ, 3.51 GHz, and 3.52 GHz from the distal, middle, and upper regions, respectively. There is a 60 MHz frequency shift, averaged from 5 data, between the distal and upper regions on the forearm. It is almost negligible compared to the frequency shift of around 250 MHz between the cases with and without a vein underneath. This indicated the different measured locations on the forearms did not have a significant effect on detecting the vessels underneath. The distinguishable resonant frequency shift was primarily due to the presence of blood vessels.

### Artery Detection.

Accidental arterial puncture often results in serious complications and patient distress. It is a prevalent and severe issue for venipuncture [17], [81], [82]. This occurrence is likely when inexperienced caregivers inadvertently mistake an artery for a vein or the distribution of vessels is not clear visually. In some cases, identifying a superficial blood vessel as an artery or vein is not easy, even for an experienced anesthetist [83]. In an emergency situation when antiepileptic drugs can only be administered intravenously for pediatric patients [84], a successful venipuncture for intravenous infusion without arterial puncture is critically important. The radial artery is one of the arteries with the most mistaken arterial punctures [85], [86], especially near the wrist [87]-[90]. Thus, the radial artery in the wrist is targeted for measurements in this study to determine if the tuned sensor can distinguish or locate the artery.

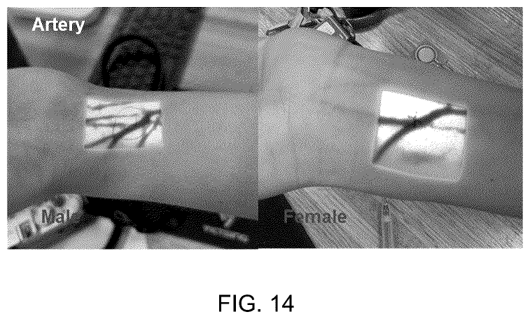

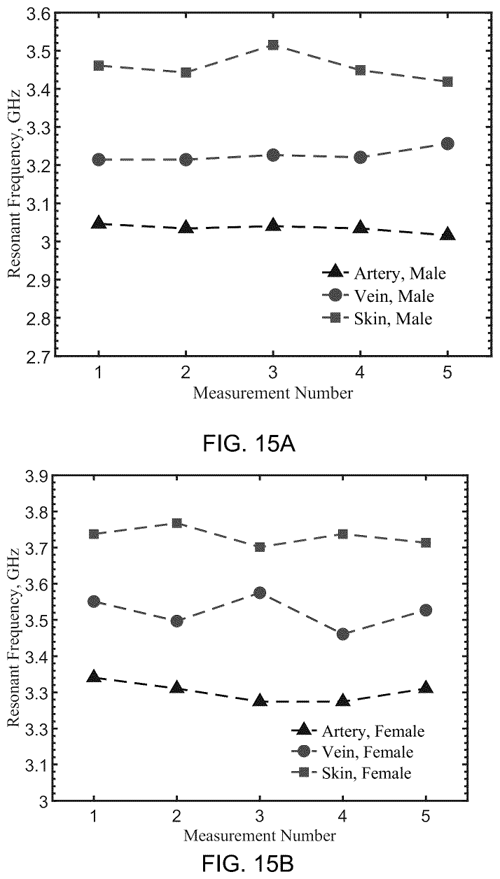

The artery, with a larger vessel diameter [87], has higher effective dielectric properties within the sensing field volume from the sensor than veins. Experiments for artery detection were conducted on both male and female subjects. A measured site was selected on the wrist artery in the left forearm for each subject, confirmed with the assistance of the NIR vein finder, as shown in FIG. 14, for both male and female subjects. Measurements were repeated five times in each location. The sensor was removed and placed again on the skin for each measurement. The resonant frequencies at the sites on the same forearm with a vein underneath, with an artery underneath, and without both are compared in FIGS. 15A and 15B for male and female subjects, respectively. On average, a 192-MHz frequency shift was observed for the male subject between the vein and artery measurements with resonant frequencies of 3.226 GHz and 3.034 GHz. An average 423-MHz frequency shift between 3.457 GHZ and 3.034 GHz for skin and artery measurements for male. For the female subject, it was 220 MHz between 3.522 GHZ and 3.302 GHz for vein and artery measurements, while a 429-MHz frequency shift between 3.731 GHz and 3.302 GHz for the skin and artery measurements. Based on different resonant frequency shifts compared to skin measurements, the results indicate that the tuned sensor can distinguish arteries and veins from the skin. The result showed the feasibility of avoiding mistaken arterial punctures.

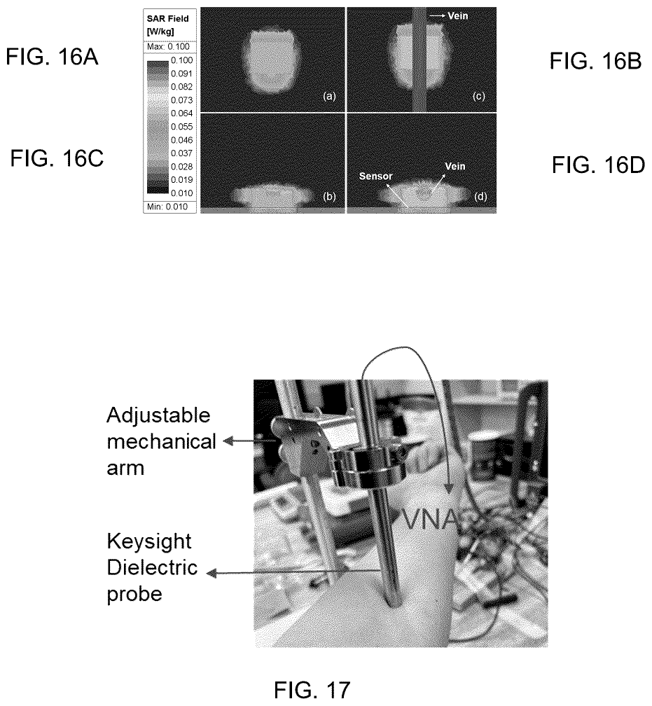

Exposure Safety. Considering the sensor on the skin radiates electrical fields into the tissues, the electromagnetic exposure safety concern should be evaluated. While it emits non-ionizing radiation without a worry for molecular changes or DNA damages that could harm biological tissues like X-rays or gamma rays [91], it has been known that high levels of RF radiation exposure may still pose health risks [92]. The rapid heating of biological tissues by RF energy has been linked to potential harm. Federal Communications Commission (FCC) mandates compliance with a Specific Absorption Rate (SAR) level of 1.6 watts per kilogram (W/kg) that is averaged over one gram of tissues [93]. The ICNIRP guidelines specify a limit of 2 W/kg averaged over 10 grams of tissues [94]. To investigate the SAR from the tuned sensor on the skin, simulations were conducted. The input power from the source was set at 5 dBm, the same as the PNA setting in the measurements. The SAR field distributions in 2-D cross-sections inside the tissues without a vein underneath are shown in FIGS. 16A and 16B as the top and side views, respectively, while FIGS. 16C and 16D show the top and side views with a vein underneath. The vein diameter is 3 mm. The SAR levels have a maximum value of 0.07 W/kg at the resonant frequency of 3.375 GHz without a vein underneath and 0.08 W/kg at 3.375 GHZ without a vein underneath. The SAR levels in both cases are far below the limits of 1.6 W/kg and 2 W/kg defined by FCC and ICNIRP. Therefore, the proposed sensors should be safe from electromagnetic field exposure. The field distributions show that the energy does not penetrate through the forearm and is limited in localized areas. Thus, there should not be a concern about the energy affecting other organs in the body.

Dielectric Property Measurements. The repeatable results herein manifest that skin with a vein underneath has a higher effective permittivity, which matches the theory as blood vessels contain higher water content than cells. To the inventors' best knowledge, there are no studies that compares effective dielectric properties on the skin with or without veins underneath. The theory and simulation conducted in this study were based on the documented dielectric properties of the skin, blood, and blood vessel wall in [57]. The documented data were averaged from multiple values obtained from separated or biopsied samples that might not resemble the effective dielectric properties on the skin. The samples were individualized, which meant they were not integrated into a single living structure and without circulating water/blood in them.

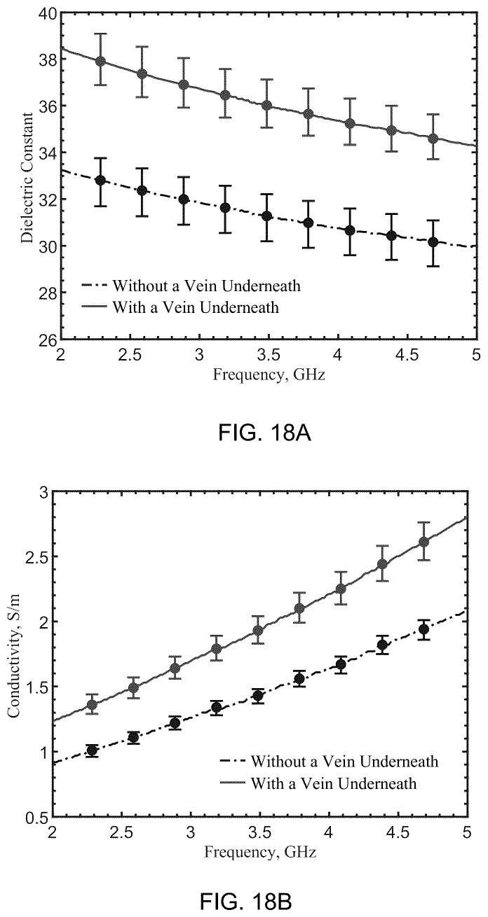

Next, direct measurements of dielectric properties on the human forearm were conducted using a coaxial probe kit (Keysight N1501A). This measurement will provide effective dielectric properties of the connected tissues in the matrices under the skin instead of separated samples from biopsies. The probe was placed on the skin of the male's forearm with and without a vein underneath, as shown in FIG. 17. The measured sites were the same as those for the resonator sensor and validated by the NIR vein finder shown in FIGS. 8A and 8B. The measured dielectric constant and conductivity are shown in FIGS. 18A and 18B. The error bars show the data ranges from five measurements. It was observed that the pressures on the skin affected the measurement results due to the changes in the vein depths caused by the pressure and compression of tissues, although the probe was pushed lightly on the skin with the intention of minimizing the data deviations from various pressures. The differences in the effective dielectric properties between the ones with and without veins underneath were distinguishable. The measured site with a vein underneath has a higher effective dielectric constant and conductivity, as expected, due to the high water content within the vein, resulting in a lower resonant frequency when the sensor is applied.

There are clear discrepancies between the documented dielectric properties from the biopsied samples [57], shown in FIGS. 3A and 3B, and the measured results on the skin, shown in FIGS. 18A and 18B. Several factors need to be considered. First, the biopsied samples only represented individual tissue properties. These samples were not the same as they were in realistic physiological conditions. Second, the values presented in were calculated as averaged values sampled from various subjects. The individual persons' conditions were unknown. The tissue compositions in individuals could play an important role in affecting these tissues' dielectric properties. Third, the samples were individualized during the microwave measurements. It is unclear if the fields from the measurements were confined within the sample and isolated from environmental effects.

The effective dielectric properties in the measurement volume are influenced by the tissue composition and also the field distributions from the probe. The variations from above mentioned effects seem to be limitless. Furthermore, in the measurements of using a coaxial probe to evaluate the effective dielectric properties, although they give a holistic view into the connected tissues, the results are person-dependent. From FIGS. 18A and 18B, the average effective dielectric constants were 36.72-34.83 in the range of 3-4.5 GHZ for the skin with a vein underneath, as compared to 57.36-54.82 for blood and 42.12-40.23 for the skin in FIGS. 3A and 3B. However, the average effective dielectric constants were 31.84-30.36 in the range of 3 4.5 GHz for the skin without a vein underneath, which was lower than 42.12-40.23 for the skin in FIGS. 3A and 3B. The variation in tissue composition, including fat layers and their thickness, capillary blood vessel densities, water content in cells and epidermis layer thickness, could affect the overall effective dielectric constants. As these factors are person-dependent and it is difficult to design a sensing device that relies on deterministic permittivity and conductivity inside the tissues, this highlights the feature of sensing the differences across the skin surface to identify the vein location. The high-quality factor resonator, together with the distinguishable difference for the skin with and without a vein underneath in FIGS. 18A and 18B, makes this detection possible without the necessity of knowing the accurate values of dielectric properties in individuals.

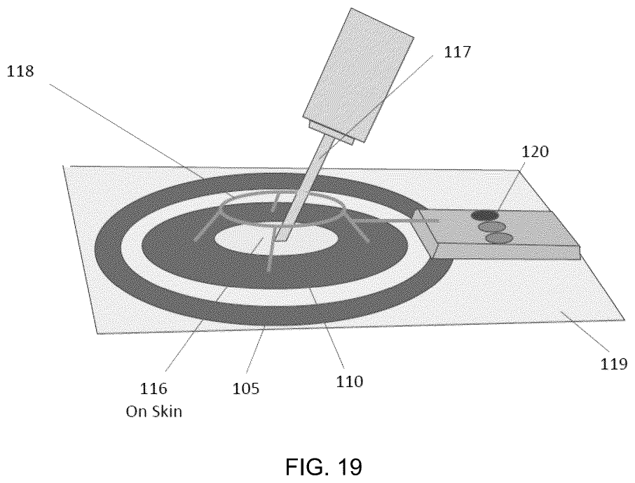

An embodiment of the present invention is shown in FIG. 19. The embodiment shown includes the sensor 100 with the loop 105 and the metal pad 110, wherein the metal pad 110 includes a hole 116 at its center that is configured to receive an injection needle 117. The skin surface of a patient is visible through the hole 116 as shown. This embodiment of the sensor 100 can include a needle holder 118 disposed on the metal pad 110 over the hole 116, wherein the needle holder 118 is configured to receive the injection needle 117 to permit the needle to move with the sensor 100 as the sensor 100 is moved over a patient's skin surface. This embodiment of the sensor 100 can also include a thin film 119 on which the sensor 100 is disposed to permit easier movement of the sensor 100 over the patient's skin surface. This embodiment can further include a visual indicator 120 that indicates when the sensor 100 is centered over a blood vessel (not shown).

An embodiment of the present invention comprises, consists essentially of, or consists of: a resonator mounted on a substrate; and a processor connected to the resonator; wherein the resonator and the processor are configured to detect at least one of resonant frequencies and the reflection coefficient magnitudes when the resonator is applied to a skin surface of a patient. In one aspect, the resonator is a tuned loop resonator comprising an outer loop and a metal pad disposed within the outer loop and concentric with the outer loop such that a gap width between the outer loop and the metal pad is constant. In another aspect, the metal pad has a hole at a center thereof, the hole configured to receive an injection needle. In another aspect, the processor uses a vector network analyzer (VNA), network analyzer (NA), and/or spectrum analyzer (SA).

By way of explanation, the present disclosure can use any of a vector network analyzer (VNA), network analyzer (NA), and/or spectrum analyzer (SA). VNA data includes phase of reflection coefficient, while a NA does not include phase data. The VNA data includes magnitude of reflection coefficient at a certain frequency. Both VNA and NA have to sweep frequencies. As such, it is possible to find the resonant frequency of a resonator by finding the minimum of reflection coefficient point.

In a spectrum analyzer, the SA sweeps frequencies and measures the power at each frequency. When a resonator is connected to a spectrum analyzer, the maximum power (peak) across the sweeping range will show the resonant frequency. Peak or maximum power is used because the reflection is at a minimum at the resonant frequency, as such, power radiates out more than other frequencies.

In another aspect, the resonator and the processor are configured to detect differences in the at least one of the resonant frequencies and the reflection coefficient magnitudes when the resonator is applied to more than one location on the skin surface. In another aspect, the resonator and the processor are configured to detect differences in the at least one of the resonant frequencies and the reflection coefficient magnitudes when the resonator is applied to the skin surface over the blood vessel and when the resonator is applied to the skin surface over tissue surrounding the blood vessel. In another aspect, the resonator and the processor are configured to detect changes in the at least one of the resonant frequencies and the reflection coefficient magnitudes over time when the resonator is applied to the skin surface. In another aspect, the resonator is applied to the skin surface over a blood vessel. In another aspect, the system further includes one or more additional resonators configurable in an array on the skin surface, permitting, e.g., detection of conditions in and along a blood vessel or focusing a field distribution into a deeper location under the skin surface.

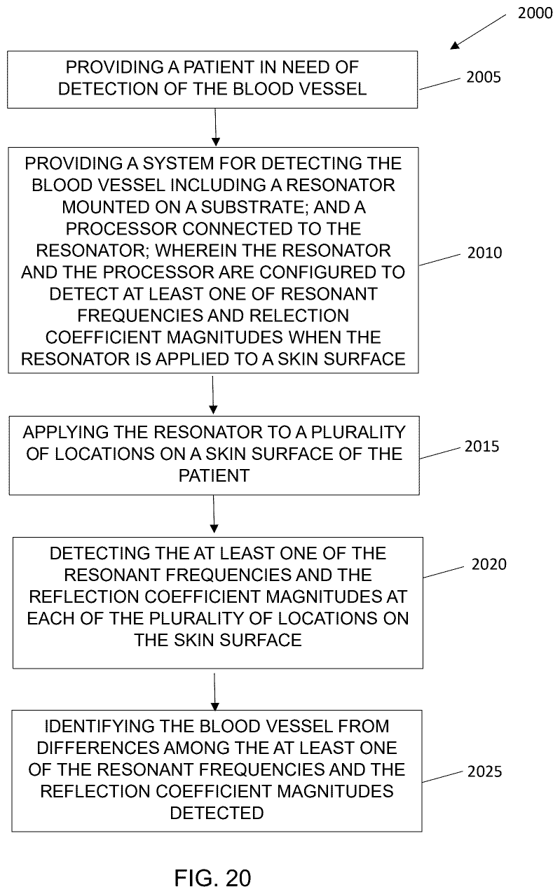

FIG. 20 shows a flowchart for a method embodiment of the present invention. The method 2000 of detecting a blood vessel comprises, consists essentially of, or consists of the blocks described herein, including block 2005, providing a patient in need of detection of the blood vessel, and block 2010, providing a system for detecting the blood vessel including a resonator mounted on a substrate; and a processor connected to the resonator; wherein the resonator and the processor are configured to detect at least one of resonant frequencies and resonant frequency magnitudes when the resonator is applied to a skin surface of the patient. Block 2015 of the method 2000 includes applying the resonator to a plurality of locations on a skin surface of the patient. Block 2020 includes detecting the at least one of the resonant frequencies and the reflection coefficient magnitudes at each of the plurality of locations on the skin surface. Block 2025 includes identifying the blood vessel from differences among the at least one of the resonant frequencies and the reflection coefficient magnitudes detected.

The present inventors demonstrate a novel noninvasive vein finder based on a tuned microwave loop resonator. It provides a low-cost, reliable, and convenient solution to address the persistent challenges in venipuncture, especially for the cases of difficult venous access. The planar loop resonator tuned with a metal pad ensures a compact design suitable for wearables and provides a high-quality factor of resonance for high sensitivity to detect permittivity changes due to the existence of veins under the skin. The utilization of low-power microwave sensing on the distinctive dielectric properties between blood vessels and tissues provides a high level of sensitivity and specificity.

Extensive simulations and experimental measurements on male and female subjects confirm the sensor's ability to detect veins with consistently distinguishable resonant frequency shifts. The repeatability across different forearm locations was validated. The capability to differentiate between arteries and veins demonstrates the robustness and potential to improve venipuncture procedures, reduce complications, and enhance patient comfort. Safety considerations for specific absorption rates are addressed by simulations showing they are below regulatory limits. Furthermore, direct measurements of dielectric properties on human forearms, which have not been documented before, validate the principle. This disclosure shows the capability and performance of noninvasive detection of vein locations by a planar loop resonator on the skin of the forearm. The validation is performed with, e.g., a vector network analyzer to ensure accuracy, however, it can also be other types of analyzers such as a spectrum analyzer. The next step will be the development of an electronic system for detecting and locking the resonance frequencies of interest when the sensor is placed on the skin.

Other embodiments of the invention address aspects of venipuncture such as patients with difficult venous access (DVA). Venipuncture, the process of drawing blood from veins, is one of the most common medical procedures. It is conducted routinely for clinical blood works, blood donations, and transfusions [95]. While the procedure is essential and widely performed, it remains challenging for a large population of people and the success is heavily dependent on the skill of healthcare providers. In the United States alone, an estimated 1.4 billion venipunctures are conducted annually, but complications and errors are common [96]. A significant challenge arises with many patients who have difficult venous access (DVA), a condition where veins are not easily visible or palpable. DVA, often defined as requiring two or more failed attempts to insert an intravenous (IV) line in arms or hands. The rates of successfully establishing an IV line vary from 53% to 86% in adults and children and drop to around 33% in infants [97].

Multiple failed venipuncture attempts are not just physically uncomfortable but also emotionally distressing for patients. Complications such as bruising, hematomas, nerve damage, or arterial punctures can occur [98]. Repeated attempts create fear or anxiety, making future procedures even more difficult. Factors like obesity, aging skin, chronic medical conditions, or needle phobia further complicate venipuncture [99]. Obesity, for example, results in deeper veins obscured by subcutaneous fat, while aging skin is often thinner and more fragile, complicating the procedure. These patient-specific challenges, combined with a global shortage of skilled phlebotomists, highlight the urgent need for tools that can improve venipuncture processes [100].

Technological advancements have attempted to address these issues. Near-infrared (NIR) imaging utilizes absorption properties of deoxygenated hemoglobin to highlight veins under the skin [101]. Commercial devices like VEINVIEWER® and ACCUVEIN® have been developed for this purpose. However, these devices are often expensive, bulky, limited to shallow veins within about 3 mm of the surface [102]. They produce noisy results. Ultrasound cross-sectional imaging into the skin can locate a deeper vein but requires specialized training to operate the transducer as the operator can only see a cross-sectional 2-D image into the tissues at any skin location. The equipment is costly making it impractical for widespread use [103]. Studies have shown that both methods have limitations [104].

Microwave sensing presents a promising alternative to these methods. Using low-power, non-ionizing radiation, this technique leverages the significant contrast in dielectric properties between blood vessels and surrounding tissues. Blood, due to its high water content, interacts differently with electromagnetic waves from skin or muscle, allowing veins to be detected by identifying the boundaries between blood and tissues. The changes in reflection coefficients and resonant frequencies of microwave interaction can be used for sensing [105]. The low power density of microwave sensing makes it safe for biological tissues. Modern advancements in RF electronics have made these systems small, energy-efficient, and cost-effective. These features enable the integration of microwave sensors into compact wearable devices, making them practical for clinical and outpatient settings.

Embodiments of the present invention include microwave sensing for vein identification applications. The sensor conforms to the human forearm and uses low-power fields to noninvasively detect veins beneath the skin. These embodiments include a noninvasive vein imager based on a tuned microwave loop resonator. It offers an affordable, reliable, and practical solution to the challenges in venipuncture particularly for patients with DVA. These embodiments feature a compact device on conformal substrate, making it suitable for raster scan applications on the skin of the forearm to identify veins. The sensor provides high sensitivity by detecting changes in tissues' dielectric properties caused by veins under the skin. Experiments targeting the median cubital veins and cephalic veins show the method feasibility to consistently detect veins with distinguishable frequency shifts.

Planar resonators are smaller compared to waveguide resonators [106], making them suitable for vein detection, especially on children's arms. Split-ring resonators (SRRs) have been widely used in planar circuits for sensing applications [107], where the coupling between the outer and inner rings creates resonance. However, these designs suffer from insertion losses and having multiple harmonics, which degrade the quality factor of the resonance of interest. SRRs can be highly sensitive to environmental changes, making them less reliable for applications on human skins. Complementary split-ring resonators (CSRRs) offer another option [108], but their indirect excitation mechanism further reduces field penetration depth, making them less sensitive in detecting veins. Planar loop resonators are more promising due to their compact size. However, conventional loop resonators face challenges in achieving proper impedance matching [105], leading to poor resonance quality and insufficient electric field penetration. While dynamic matching circuits can address this issue, they increase the device footprint and complicate its design. To overcome these challenges, previously [109] a self-tuned method by embedding a concentric metal pad within the loop resonator was introduced. This design provided distributed capacitance and mutual inductance through the gap between the loop and the metal pad, allowing impedance matching without additional external tuning circuits. The result was a significant improvement in resonance performance, enabling compact and efficient designs suitable for near-field sensing applications [110]-[117]. Embodiments of the present invention adapt the tuned loop resonator for imaging veins beneath the skin.

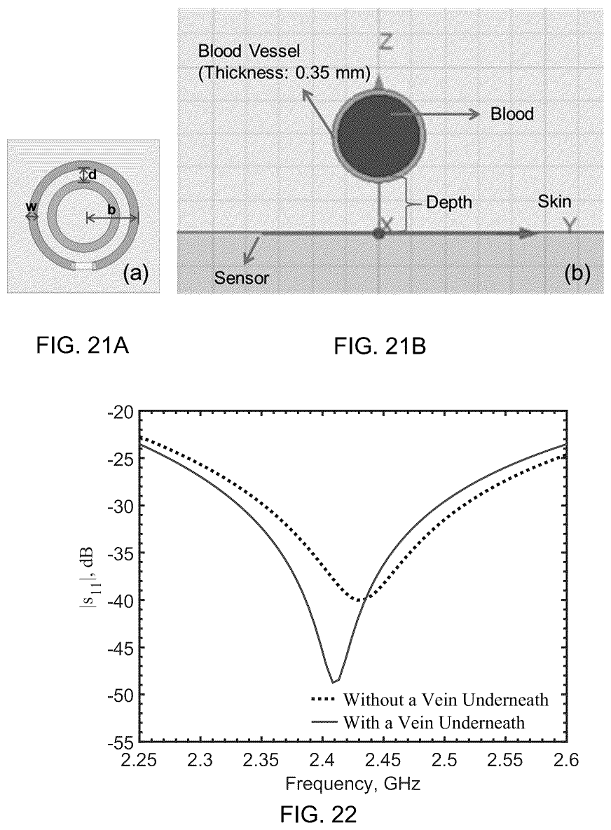

In addition, embodiments of the invention include a design that replaces the center pad with a complete ring, which not only tunes the loop impedance but also preserves access by the needle for venipuncture. The complete inner ring allows for additional distributed capacitance along the ring and fields across the gap and ring improving sensitivity. To enhance comfort and adaptability, the resonator was designed on a flexible poly-imide film (DuPont™ Pyralux® FR9220R) with a thickness of 76 μm and a dielectric constant of 3.2. The copper layer has a thickness of 70 μm. The sensor design is shown in FIG. 21A.

The simulation model, shown in FIG. 21B, includes skin, vessel wall, and blood. The vessel wall was modeled with a thickness of 0.35 mm [118], [119]. Dielectric properties for the skin, blood, and vessel walls were from a documented library [120].

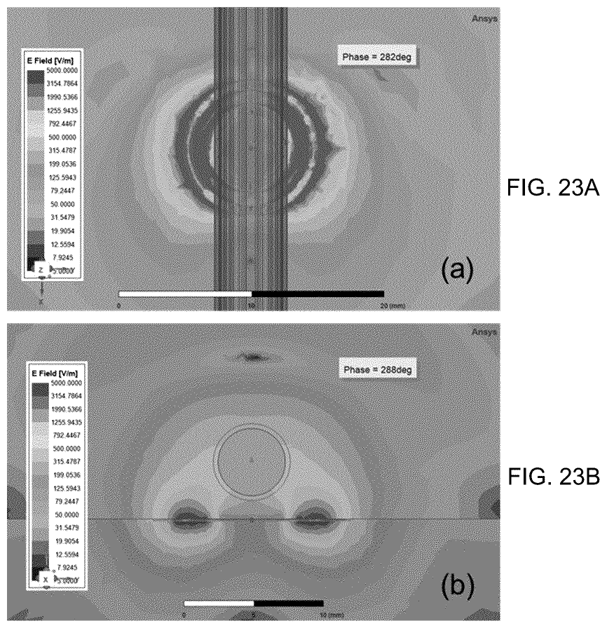

The loop has a radius b of 5.2 mm, a metal width w of 0.7 mm, and a gap d of 1.5 mm between the loop and the tuning ring, optimizing a resonance at 2.408 GHz with |s₁₁| of 48.77 dB with a vein present at a depth of 3 mm and the diameter of the vein is 2.9 mm. FIG. 22 presents the simulated reflection coefficients with and without a vein beneath the sensor. For the case without a vein underneath, the resonance occurred at 2.433 GHz with |s₁₁| of 39.99 dB. A frequency shift of 25 MHz was achieved. The strong resonance with or without vein underneath, indicated by the similar nulls in |s₁₁|, provides localized electric fields that penetrate sufficiently into the skin, allowing the sensor to detect changes in effective permittivity caused by the presence of a vein, as shown in FIG. 23.

### Subcutaneous Vein Scanning.

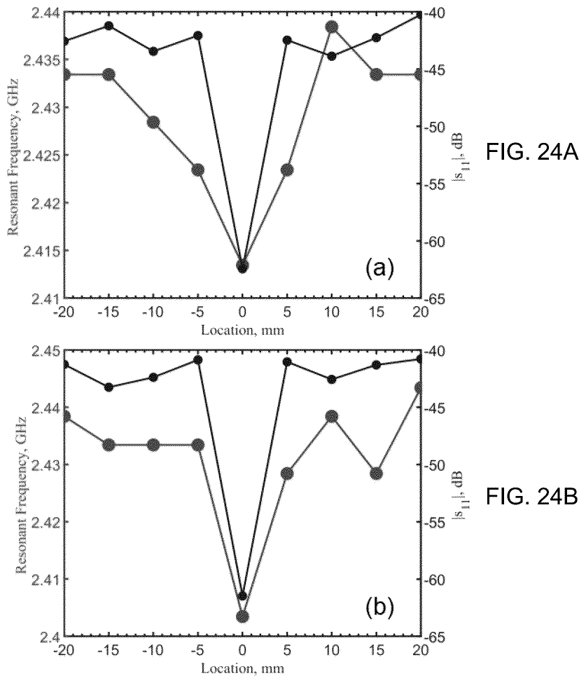

Superficial veins, such as the cephalic vein (CV) and median cubital vein (MCV), are commonly used for venipuncture [102]. The depth and diameter of these veins vary among individuals. For the CV, the depth typically ranges from 1.7 to 2.1 mm, with diameters between 1.7 and 2.9 mm. For the MCV, the depth ranges from 1.7 to 3 mm, and the diameter spans 2.3 to 24.9 mm [102], [121]. To validate the concept, two challenging cases were simulated: a CV with a diameter of 1.7 mm and a depth of 2.1 mm, and an MCV with a diameter of 0.3 mm and a depth of 3 mm. A raster scan by the sensor with 5-mm scanning steps across the skin shows a clear indication of vein location (at the center, 0 mm) by the resonant frequencies and |s₁₁| in FIGS. 24A and 24B. To evaluate the electric field penetration by the tuned sensor and determine the maximum detectable vein depth, the depth of the vein in simulations gradually increased. For the MCV, the sensor could successfully detect it at a depth of 6 mm, with a resonant frequency of 2.423 GHz and |s₁₁| of 44 dB, compared to 2.4334 GHZ and |s₁₁| of 39.99 dB in the absence of a vein. Similarly, the CV was detectable at a maximum depth of 5 mm, with a resonant frequency of 2.423 GHz and |s₁₁| of −42 dB. While these depths exceed the typical values for MCV and CV documented in the literature, the results demonstrated the sensor's capability for detecting deeper veins under challenging conditions.

In practical applications, the vein location may require an image map with a 2-dimensional raster scan on the targeted tissue area by moving the sensor. The image can be obtained with the tuned loop resonator switched temporally to acquire individual pixel data. Based on the reflection coefficients, heatmaps can be generated to illustrate the location of the vein. A discrete raster scan was conducted with the tuned loop to validate this concept. An MCV vessel with a radius of 1.15 mm was placed at X=0 at a depth of 3 mm. Each pixel was 5×5 mm². The total scanning area was 20×40 mm² with 5×9 pixels.

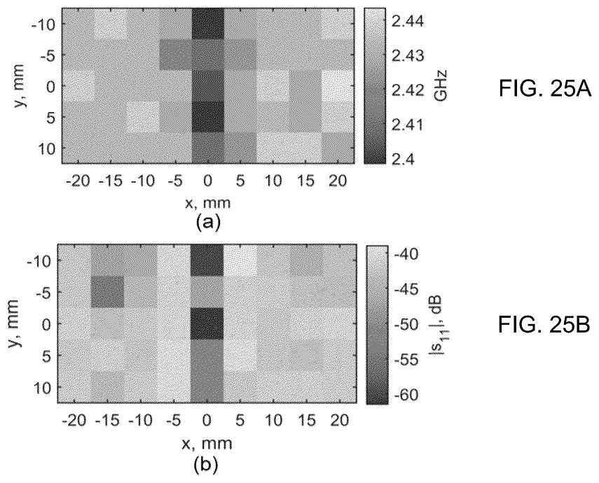

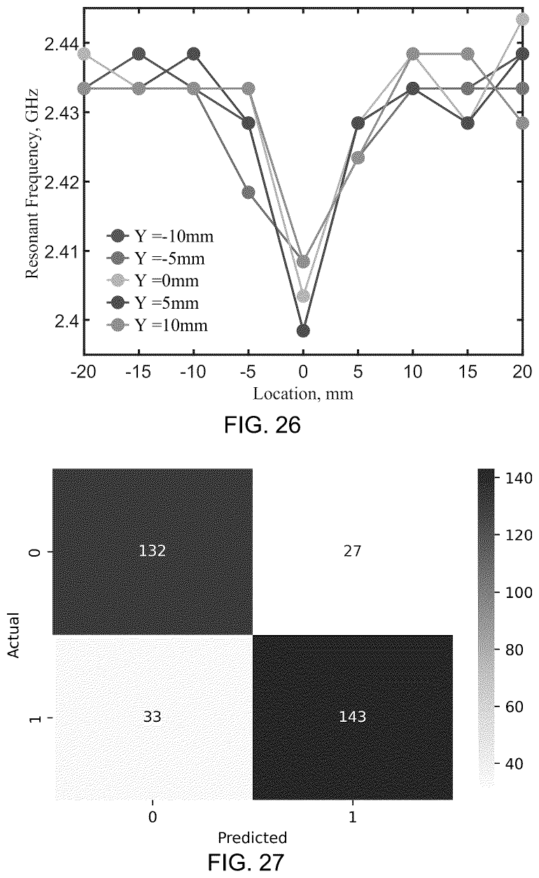

FIG. 25A shows the heatmap of the resonant frequencies, represented in pixels with different color scales normalized from 2.4 to 2.442 GHz. Similarly, FIG. 25B shows the heatmap of the |s₁₁| and scales normalized from 60 to 39 dB. It clearly shows that the location of the vessel is at X=0 mm, where the resonant frequencies and |s₁₁| are lower, representing darker blue pixels. The neighbor pixels and other pixels away from the center show resonant frequencies in the range of 2.42 to 2.44 GHz while |s₁₁| in the range of 39 to 50 dB. The reflection coefficients at Y from −10 mm to +10 mm of this scanned map are compared and shown in FIG. 26. The curves clearly indicate that the distinguished frequency shifts with or without a vessel underneath.

### Data Analysis.

The previous tuned microwave loop resonator for vein detection was investigated and comprehensive simulation models were developed representing various scenarios. A concern is the potential variability in dielectric constants of human tissues, as these properties exist within ranges rather than as definite values. This variability could introduce measurement inconsistencies, with discrepancies arising from simulation models and individual subject characteristics. To address this challenge, the inventors established two distinct datasets, each containing data points characterized by two features: frequency and |s₁₁| parameters. The inventors framed the analysis as a binary classification problem (0 or 1) to determine the presence of a vein beneath the sensor. The first dataset comprised 63 data points derived from simulation models described in previous sections. Based on the described hereinabove, a second dataset contained 335 data points. A clear distinction between these datasets was observed in their frequency distributions, with the simulation dataset exhibiting an average frequency of 2.43 GHZ compared to 3.41 GHz in the experimental dataset. The inventors normalized each dataset independently to evaluate the relationship between these datasets using machine learning techniques. The simulation dataset served as training data, while the experimental dataset functioned as test data.

A decision tree model was employed for its interpretability in small-scale classification tasks. Grid search optimization revealed that the |s₁₁| parameter contributed minimally to prediction accuracy, resulting in a model that relied exclusively on frequency as the predictive feature, with a tree depth of one. This streamlined model achieved 82.09% accuracy on the experimental data. Further analysis of the decision tree parameters yielded a threshold value of 0.536. When the normalized frequency falls below this threshold, the model indicates a high probability of vein detection. This threshold-despite being derived from simulation data—demonstrated high accuracy when applied to experimental measurements. With additional subject data collection, further improvements in real-world applicability are anticipated, potentially mitigating the challenges associated with the variable dielectric properties of human tissues. FIG. 27. shows a confusion matrix that illustrates the performance of the decision tree classification model when applied to the experimental dataset. The model achieved 82.09% overall accuracy in distinguishing between the presence (1) and absence (0) of veins beneath the microwave loop resonator sensor.

The resonator sensor detects the reflection coefficient |s₁₁| within a range of frequency of interest. The magnitude and phase of |s₁₁| as a function of frequency are recorded. The frequency with the minimum |s₁₁| is treated as the resonant frequency. The resonant frequency and its respective magnitude at a location on the skin are used to generate two images. One image contains pixels of the resonant frequencies, and one image contains pixels of the |s₁₁|. The resonant-frequency image should indicate the vein location as a line of pixels when the vein is not deep because the resonant frequencies with a vein under the skin should be lower than the neighbor pixels. The |s₁₁| image should also indicate the vein location when the vein is not deep as the resonance with the vein underneath the skin should be better than the neighbor pixels.

However, two factors may affect the results. (1) The resonator sensor has its specific electrical field distributions inside the tissues under the skin. The field distributions depend on individual person's tissue composition such as the skin thickness, fat and water contents in the tissues, bone, tissue density, and capillary blood vessels. The electrical fields, due to its variations in distribution, produce variations in the reflection coefficients. (2) The depth of field penetration into the tissues varies depending on the transmission power from the resonator sensor. Although the reflection coefficient measurement calibrates the output power and it should not be affected by radiation power in the tissues, stronger powers penetrate deeper into the tissues and the contribution of dielectric parameters in deeper tissues can affect the effective dielectric parameters sensed by the resonator sensor affecting its reflection coefficient.

The sensor reflection coefficient is more dominated by dielectric parameters near the skin than deeper tissues. As the fields penetrate deeper, the amplitudes of the electrical fields decay and the dielectric parameters from the deeper tissues become less influential to the reflection coefficient. These two factors become more clear when the vein is deeper under the skin because the resonance effect from the water in the vein becomes less prominent. One clear evidence observed is that the resonant frequency with a vein does not monotonically varies when the vein depth increases. When the vein depth is deep, clear maps of resonant frequencies and |s₁₁| may be more difficult to obtain.

Combining the two data sets to generate a more robust map helps recognition of the vein location. Both data sets are normalized for their measured minimum and maximum ranges. The normalization ranges between 0 and 1, with 0 being the lowest resonant frequency and 1 being the highest resonant frequency in the resonant-frequency map, and 0 being the lowest |s₁₁| and 1 being the highest |s₁₁| in the |s₁₁| map, respectively. Two maps are then generated according to the scale from 0 to 1. A weighting value w is used to emphasize the influence of a specific map. The value w is between 0.01 and 0.99. One map is given x while the other map is given 1-w. An integrated map is generated with each pixel containing the sum of the pixel value times w from one map and the pixel value times (1-w) from the other map. If the resonant-frequency map clearly indicates a line of pixels with values much lower than others, the resonant-frequency map is given a larger value w. If the |s₁₁| map clearly indicates a line of pixels with values much lower than others, the |s₁₁|map is given a larger value w, while the resonant-frequency map is given a smaller value of (1-w). A series of integrated maps are generated with a series of varying w values. Then the maps are analyzed to compare contrasts. The highest contrast map gives the clearer vein location, as a line, and determines the optimal w value.

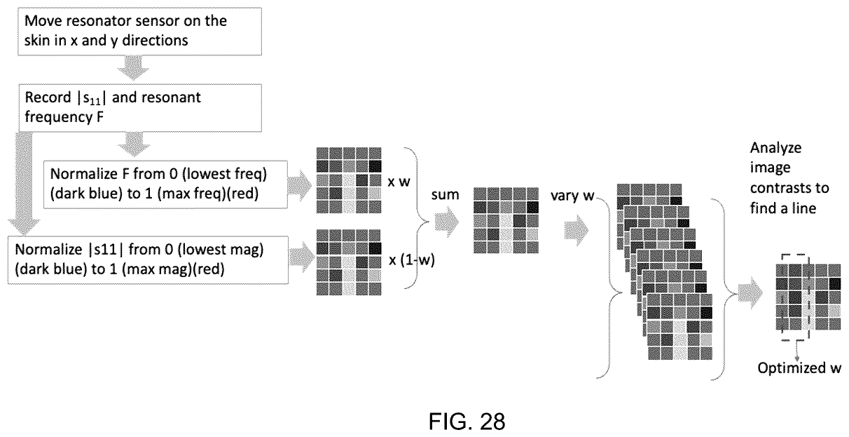

Machine learning techniques were used to differentiate the contrasts in an image map. These machine learning techniques are trained with training data and compared with the ground truth. The machine learning techniques can provide insight to the image contrasts and automatically generate the optimal w value when using the resonator sensor for raster scanning. FIG. 28 shows an example of a machine learning technique to train contrast recognition.

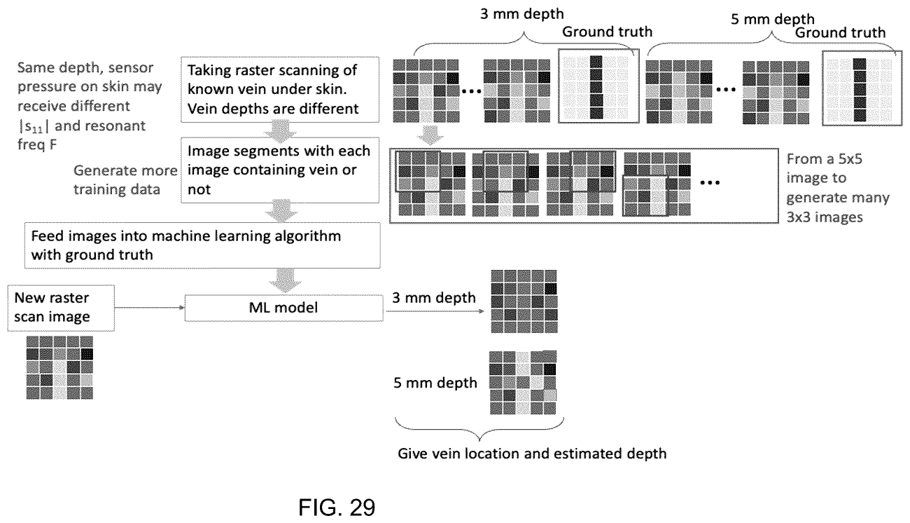

The machine learning technique to help recognition of the vein is conducted through training data from various scans from different subjects. Because the pressure of the sensor on the skin can also affect the reflection coefficients, various scans on the same person are conducted for training purpose. The pixel maps are collected for known vein locations. The depths of the veins can be verified by an ultrasound transducer. Because machine learning accuracy can be improved by more training data, the maps are segmented into smaller maps for training. The maps are also rotated to change the vein locations. Ground truth for each map can contain vein locations in certain pixels or without vein pixels. FIG. 29 shows another example of a machine learning technique to train contrast recognition.

Clinically, hospitals have been using ultrasound imagers to probe vein locations. The images are typically fuzzy because the pressure of the ultrasound transducer on the skin moves vein and tissues under the skin. Phlebotomist looks at the cross-sectional images by moving the ultrasound transducer on the skin to judge the potential vein location. Because the black-and-white cross-sectional images show only the depth information, it is often difficult to clear identify the vein medial/lateral and proximal/distal location without extensive training.

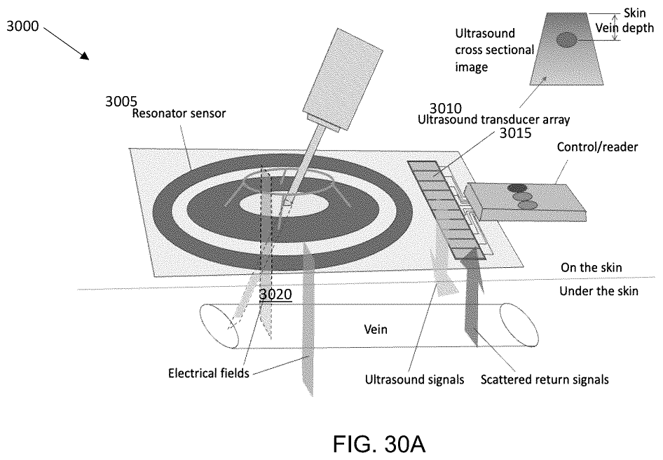

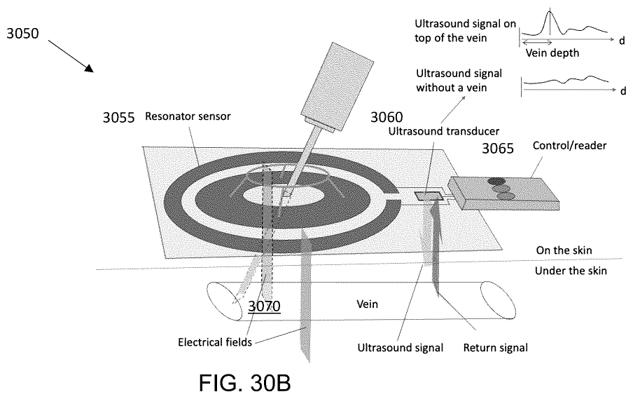

The integration of the resonator sensor with ultrasound transducer array can enhance the vein location identification. The resonator sensor first identifies the vein in terms of medial/lateral and proximal/distal location, and at the same time the ultrasound cross-sectional image indicates the vein depth under the skin. In the figure, the transducer is on one side of the flexible substrate, but it can be on the other side. The transducer array transmits ultrasound signals at 2-15 MHz into the arm. The returned signals scattered by different types of tissues receive by the transducer. The return signals can be used to construct the cross-sectional image as the timing of the return signal indicate the depth while different pixels provide spatial resolution. The resonator sensor utilizes much high frequencies at GHz ranges and electrical fields for sensing. The ultrasound transducer utilizes signal frequencies in the range of MHz and mechanical vibrational waves. These two signals will not have interference or cross talk. Because the electrical fields 3020 already identify the vein location, the transducer may not need to be an array. A single transducer can be used to identify the vein depth by comparing the return signals on top of a vein and without a vein. FIGS. 30A and 30B show configurations of the resonator sensor integrated with one or more ultrasound transducers. FIG. 30A shows an integrated apparatus 3000 that includes a resonator sensor 3005, an ultrasound transducer array 3010, and an array processor 3015. Also shown is a blood vessel 3120 (which can be a vein, an artery, capillary beds, etc.) over which the integrated apparatus is positioned. FIG. 30B shows an integrated apparatus 3050 that includes a resonator sensor 3055, an ultrasound transducer array 3060, and an array processor 3065. Also shown is a blood vessel 3120 over which the integrated apparatus is positioned.

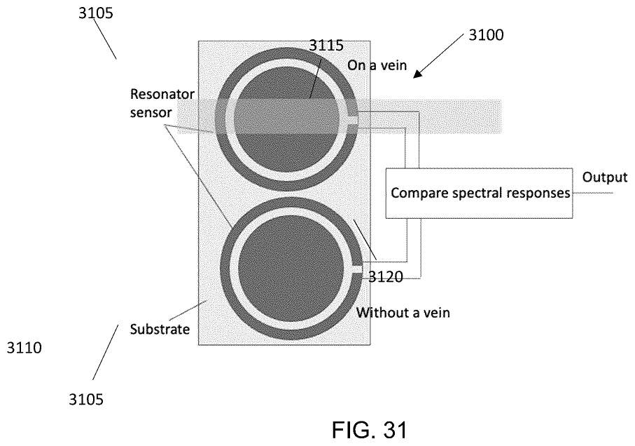

Experiments have been conducted to examine sensor outputs on top of a major blood vessel (vein or artery) and without a blood vessel. The spectral responses are different in terms of resonant frequency and |s₁₁|. The sensor with two resonators on the same substrate can be used to compare the cases with and without a blood vessel 3120 underneath the skin. The reason for integration is that the tissue properties in the nearby area under the skin should be similar, except the difference of having a blood vessel under one sensor. The spectral responses within the frequency range of interest are compared to identify resonant frequency shift. The differential comparison makes clear the existence of a blood vessel. The figure is the top view of the sensors and substrate. FIG. 31 shows a two-resonator configuration for such a differential comparison. The Two-resonator configuration 3100 includes a sensor with two resonators 3105a, 3105b; a substrate 3100 on which the two resonators 3105a, 3105b are mounted; a blood vessel 3115 over which one of the resonators, here resonator 3105a, is located. Spectral responses from the two resonators 3105a, 3105b are compared by processor 3120.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

[1] W. H. Organization, “Who guidelines on drawing blood: best practices in phlebotomy,” 2010. [Online]. Available: www.who.int/iris/handle/10665/44294.
[2]C. Cairns and K. Kang, “National hospital ambulatory medical care survey: 2021 emergency department summary tables,” Tech. Rep., 2021. [Online]. Available: ftp.cdc.gov/pub/Health_Statistics/NCHS/Dataset_Documentation/NHAMCS/doc21-ed-508.pdf.
[3]L. L. Kuensting, S. DeBoer, R. Holleran, B. L. Shultz, R. A. Steinmann, and J. Venella, “Difficult venous access in children: Taking control,” Journal of emergency nursing, vol. 35, no. 5, pp. 419-424, Sep. 1, 2009.
[4]J. M. F. MD, N. E. P. MD, A. K. A. MD, and M. M. Ku, Bon S., “Riskfactors associated with difficult venous access in adult ed patients,” The American journal of emergency medicine, vol. 32, no. 10, pp. 1179-1182, Oct. 1, 2014.
[5]P. J. Carr, J. C. R. Rippey, C. A. Budgeon, M. L. Cooke, N. Higgins, and C. M. Rickard, “Insertion of peripheral intravenous cannulae in the emergency department: Factors associated with first-time insertion success,” The journal of vascular access, vol. 17, no. 2, pp. 182-190, Mar. 2016.
[6]N. J. Cuper, J. C. de Graaff, A. T. H. van Dijk, R. M. Verdaasdonk, D. B. M. van der Werff, and C. J. Kalkman, “Predictive factors for difficult intravenous cannulation in pediatric patients at a tertiary pediatric hospital,” Pediatric anesthesia, vol. 22, no. 3, pp. 223-229, Mar. 2012.
[7]A. F. Jacobson and E. H. Winslow, “Variables influencing intravenous catheter insertion difficulty and failure: An analysis of 339 intravenous catheter insertions,” Heart & lung, vol. 34, no. 5, pp. 345-359, Sep. 1, 2005.
[8]A. F. Jacobson, “Intradermal normal saline solution, self-selected music, and insertion difficulty effects on intravenous insertion pain,” Heart & lung, vol. 28, no. 2, pp. 114-122, Mar. 1, 1999.
[9]F. Lapostolle, J. Catineau, B. Garrigue, V. Monmarteau, T. Houssaye, I. Vecci, V. Treoux, B. Hospital, N. Crocheton, And F. Adnet, “Prospective evaluation of peripheral venous access difficulty in emergency care,” Intensive care medicine, vol. 33, no. 8, pp. 1452-1457, Aug. 1, 2007.
[10]M. O'Neill, M. Dillane, and N. F. Hanipah, “Validating the difficult intravenous access clinical prediction rule,” Pediatric emergency care, vol. 28, no. 12, pp. 1314-1316, Dec. 2012.
[11]M. W. Riker, C. Kennedy, B. S. Winfrey, K. Yen, and M. D. Dowd, “Validation and refinement of the difficult intravenous access score: A clinical prediction rule for identifying children with difficult intravenous access,” Academic emergency medicine, vol. 18, no. 11, pp. 1129-1134, Nov. 2011.
[12]M. M. Witting, Michael D., “Iv access difficulty: Incidence and delays in an urban emergency department,” The Journal of emergency medicine, vol. 42, no. 4, pp. 483-487, Apr. 1, 2012.
[13]K. Yen, A. Riegert, and M. Gorelick, “Derivation of the diva score: A clinical prediction rule for the identification of children with difficult intravenous access,” Pediatric emergency care, vol. 24, no. 3, pp. 143-147, Mar. 2008.
[14]R. A. Lininger, “Pediatric peripheral i.v. insertion success rates,” Pediatric nursing, vol. 29, no. 5, pp. 351-354, Sep. 2003.
[15]K. Black, M. Pusic, D. Harmidy, and D. McGillivray, “Pediatric intravenous insertion in the emergency department: Bevel up or bevel down?” Pediatric emergency care, vol. 21, no. 11, pp. 707-711, Nov. 2005.
[16]P. T. Gilchrist and B. Ditto, “The effects of blood-draw and injection stimuli on the vasovagal response,” Psychophysiology, vol. 49, no. 6, pp. 815-820, Jun. 2012.
[17]N. L. Moureau, N. Trick, T. Nifong, C. Perry, C. Kelley, R. Carrico, M. Leavitt, S. M. Gordon, J. Wallace, M. Harvill, C. Biggar, M. Doll, L. Papke, L. Benton, and D. A. Phelan, “Vessel health and preservation (part 1): A new evidence-based approach to vascular access selection and management,” The Journal of Vascular Access, vol. 13, no. 3, pp. 351-356, Jul. 1, 2012.
[18]C. Jbeili, C. Penet, P. Jabre, L. Kachout, S. Schvahn, A. Margenet, J. Marty, and X. Combes, “Out-of-hospital management characteristics of severe obese patients,” Annales françaises d′anesthésie et de réanimation, vol. 26, no. 11, pp. 921-926, Nov. 2007.
[19]P. Juvin, A. Blarel, F. Bruno, and J.-M. Desmonts, “Is peripheral line placement more difficult in obese than in lean patients?” Anesthesia and analgesia, vol. 96, no. 4, p. 1218, Apr. 2003.
[20]O. O. Nafiu, C. Burke, A. Cowan, N. Tutuo, S. Maclean, And K. K. Tremper, “Comparing peripheral venous access between obese and normal weight children,” Pediatric anesthesia, vol. 20, no. 2,pp. 172-176, Feb. 2010.
[21]L. Dougherty, “Intravenous therapy in older patients,” Nursing standard, vol. 28, no. 6, pp. 50-58, Oct. 9, 2013.
[22]L. Brannam, M. Blaivas, M. Lyon, and M. Flake, “Emergency nurses' utilization of ultrasound guidance for placement of peripheral intravenous lines in difficult-access patients,” Academic emergency medicine, vol. 11, no. 12, pp. 1361-1363, Dec. 2004.
[23]F. B. Chiao, F. Resta-Flarer, J. Lesser, J. Ng, A. Ganz, D. Pino-Luey, H. Bennett, C. Perkins, and B. Witek, “Vein visualization:patient characteristic factors and efficacy of a new infrared vein finder technology,” British journal of anaesthesia: BJA, vol. 110, no. 6, pp. 966-971, Jun. 2013.
[24]P. Larsen, D. Eldridge, J. Brinkley, D. Newton, D. Goff, T. Hartzog, N. Saad, and R. Perkin, “Pediatric peripheral intravenous access: Does nursing experience and competence really make a difference?” Journal of infusion nursing, vol. 33, no. 4, pp. 226-235, Jul. 2010.
[25]D. Millam and L. Hadaway, “On the road to successful iv starts,” Nursing, vol. 30, pp. 34-48, May, 2000.
[26]C. Sonmez, U. Yildiz, N. Akkaya, and F. Taneli, “Preanalytical phase errors: Experience of a central laboratory,” Cureus, vol. 12, no. 3, p. e7335, Mar. 20, 2020.
[27]O. Y. Buowari, “Complications of venepuncture,” Advances in bio-science and biotechnology, vol. 4, no. 1, pp. 126-128, 2013.
[28]R. Serra, N. Ielapi, A. Barbetta, G. Buffone, S. Mellace, E. Bevacqua, L. Traina, G. D. Mizio, S. D. Franciscis, and V. Gasbarro, “Adverse complications of venipuncture: a systematic review,” Acta phlebologica, vol. 19, no. 1, Apr. 1, 2018.
[29]H. J. Galena, “Complications occurring from diagnostic venipuncture,” J Fam Pract, vol. 34, no. 5, pp. 582-584, 1992.
[30]A. Sabri, J. Szalas, K. S. Holmes, L. Labib, and T. Mussivand, “Failed attempts and improvement strategies in peripheral intravenous catheterization,” Bio-medical materials and engineering, vol. 23, no. 1-2, pp. 93-108, 2013.
[31]B. C. MD, S. T. MD, and G. W. H. MD, “Predictors of successin nurse-performed ultrasound-guided cannulation,” The Journal of emergency medicine, vol. 33, no. 4, pp. 401-405, Nov. 1, 2007.
[32]N. L. Panebianco, J. M. Fredette, D. Szyld, E. B. Sagalyn, J. M.Pines, and A. J. Dean, “What you see (sonographically) is what you get: Vein and patient characteristics associated with successful ultrasound-guided peripheral intravenous placement in patients with difficult access,” Academic emergency medicine, vol. 16, no. 12, pp. 1298-1303, Dec. 2009.
[33]T. G. Costantino, A. K. Parikh, W. A. Satz, and J. P. Fojtik, “Ultrasonography-guided peripheral intravenous access versus traditional approaches in patients with difficult intravenous access,” Annals of emergency medicine, vol. 46, no. 5, pp. 456-461, Nov. 2005.
[34]J. S. Donaldson, F. P. Morello, J. J. Junewick, J. C. O'Donovan, and J. Lim-Dunham, “Peripherally inserted central venous catheters: Us-guided vascular access in pediatric patients,” Radiology, vol. 197, no. 2, pp. 542-544, Nov. 1, 1995.
[35]V. P. Zharov, S. Ferguson, J. F. Eidt, P. C. Howard, L. M. Fink, and M. Waner, “Infrared imaging of subcutaneous veins,” Lasers in surgery and medicine, vol. 34, no. 1, pp. 56-61, Jan. 2004.
[36]R. A. WEISS and M. P. GOLDMAN, “Transillumination mapping prior to ambulatory phlebectomy,” Dermatologic surgery, vol. 24,no. 4, pp. 447-450, Apr. 1998.
[37]R. W. Franz, J. F. Hartman, and M. L. Wright, “Treatment of varicose veins by transilluminated powered phlebectomy surgery: A 9-year experience,” The International journal of angiology, vol. 21, no. 4,pp. 201-208, Dec. 1, 2012.
[38]H. Haxthausen, “Infrared photography of subcutaneous veins: Demonstration of concealed varices in ulcer and eczema of the leg,” British journal of dermatology (1951), vol. 45, no. 12, pp. 506-511, Jan. 1, 1933.
[39]A. Roggan, M. Friebel, K. Do\ddot{ } rschel, A. Hahn, and G. Mu\ddot{ } ller, “Optical properties of circulating human blood in the wavelength range 400-2500 nm,” Journal of Biomedical Optics, vol. 4, no. 1, pp. 36-46, Jan. 1999.
[40]L. L. Chapman, B. Sullivan, A. L. Pacheco, C. P. Draleau, and B. M. Becker, “Veinviewer-assisted intravenous catheter placement in a pediatric emergency department,” Academic emergency medicine, vol. 18, no. 9, pp. 966-971, Sep. 2011.
[41]“Accuvein® vein visualization system.” [Online]. Available: www.accuvein.com/vein-visualization-system/.
[42]N. J. Cuper, R. M. Verdaasdonk, R. de Roode, K. M. K. de Vooght, M. A.
Viergever, C. J. Kalkman, and J. C. de Graaff, “Visualizing veins with near-infrared light to facilitate blood withdrawal in children,” Clinical pediatrics, vol. 50, no. 6, pp. 508-512, Jun. 2011.
[43]K. Mukai, Y. Nakajima, T. Nakano, M. Okuhira, A. Kasashima, R. Hayashi, M. Yamashita, T. Urai, and T. Nakatani, “Safety of venipuncture sites at the cubital fossa as assessed by ultrasonography,” Journal of Patient Safety, vol. 16, no. 1, pp. 98-105, Mar. 2020.
[44]S. J. Curtis, W. R. Craig, E. Logue, B. Vandermeer, A. Hanson, and T. Klassen, “Ultrasound or near-infrared vascular imaging to guide peripheral intravenous catheterization in children: a pragmatic randomized controlled trial,” Canadian Medical Association journal (CMAJ), vol. 187, no. 8, pp. 563-570, May 19, 2015.
[45]J. M. Leipheimer, M. L. Balter, A. I. Chen, E. J. Pantin, A. E. Davidovich, K. S. Labazzo, and M. L. Yarmush, “First-in-human evaluation of a hand-held automated venipuncture device for rapid venous blood draws,” Technology (Singapore), vol. 7, no. 3-4, pp. 98-107, Sep. 1, 2019.
[46]J.-C. Chiao, C. Li, J. Lin, R. H. Caverly, J. C. M. Hwang, H. Rosen, and A. Rosen, “Applications of microwaves in medicine,” IEEE journal of microwaves, vol. 3, no. 1, pp. 134-169, Jan. 2023.
[47]J.-C. Chiao, S. Bing, and K. Chawang, “Review on noninvasive radio-frequency sensing for closed-loop body health management,” in 2021 IEEE International Symposium on Radio-Frequency Integration Technology (RFIT). Piscataway: IEEE, Aug. 25, 2021, pp. 1-3.
[48]M. P. Robinson, J. Clegg, and D. A. Stone, “A novel method of studying total body water content using a resonant cavity: experiments and numerical simulation,” Physics in medicine & biology, vol. 48,no. 1, pp. 113-125, Jan. 7, 2003.
[49]D. A. Stone and M. P. Robinsons, “Total body water content observations using cavity-perturbation techniques,” in 2003 High Frequency Postgraduate Student Colloquium (Cat. No. 03TH8707), 2003, pp. 31-34.
[50]A. W. Kraszewski, S. O. Nelson, and T. S. You, “Use of a microwave cavity for sensing dielectric properties of arbitrarily shaped biological objects,” IEEE Transactions on Microwave Theory and Techniques, vol. 38, no. 7, pp. 858-863, Jul. 1990.
[51]M. S. Venkatesh and G. S. V. Raghavan, “overview of dielectric properties measuring techniques,” Canadian biosystems engineering, vol. 47, p. 7, 2005.
[52]K. Aydin, I. Bulu, K. Guven, M. Kafesaki, C. M. Soukoulis, and E. Ozbay, “Investigation of magnetic resonances for different split-ring resonator parameters and designs,” New journal of physics, vol. 7,no. 1, p. 168, 2005.
[53]M. Baghelani, Z. Abbasi, M. Daneshmand, and P. E. Light, “Non-invasive continuous-time glucose monitoring system using a chipless printable sensor based on split ring microwave resonators,” Scientific reports, vol. 10, no. 1, p. 12980, Jul. 31, 2020.
[54]G. Ekinci, A. Calikoglu, S. N. Solak, A. D. Yalcinkaya, G. Dundar, and H. Torun, “Split-ring resonator-based sensors on flexible substrates forglaucoma monitoring,” Sensors and Actuators A: Physical, vol. 268, pp. 32-37, 2017.
[55]H. Choi, J. Naylon, S. Luzio, J. Beutler, J. Birchall, C. Martin, and A. Porch, “Design and in vitro interference test of microwave noninvasive blood glucose monitoring sensor,” IEEE Transactions on Microwave Theory and Techniques, vol. 63, no. 10, pp. 3016-3025, 2015.
[56]J. Kilpijarvi, J. Tolvanen, J. Juuti, N. Halonen, and J. Hannu, “A non-invasive method for hydration status measurement with a microwave sensor using skin phantoms,” IEEE Sensors Journal, vol. 20, no. 2,pp. 1095-1104, Jan. 15, 2020.
[57]D. Andreuccetti, R. Fossi, and C. Petrucci, “An internet resource for the calculation of the dielectric properties of body tissues in the frequency range 10 Hz 100 GHz,” niremf.ifac.cnr.it/tissprop/, 1997, iFAC-CNR, Florence (Italy), Based on data published by C.Gabriel et al.in 1996.
[58]S. Bing, K. Chawang, and J. C. Chiao, “A self-tuned method for impedance-matching of planar-loop resonators in conformable wearables,” Electronics (Basel), vol. 11, no. 17, p. 2784, Sep. 4 2022.
[59]J. Wei, “Distributed capacitance of planar electrodes in optic and acoustic surface wave devices,” IEEE journal of quantum electronics, vol. 13, no. 4, pp. 152-158, Apr. 1977.
[60]F. Maradei and S. Caniggia, Appendix A: Formulae for Partial Inductance Calculation, ser. Signal Integrity and Radiated Emission of High-Speed Digital Systems. Chichester, UK: John Wiley & Sons, Ltd, Nov. 14 2008, pp. 481-486.
[61]S. Bing, K. Chawang, and J. C. Chiao, “A flexible tuned radio-frequency planar resonant loop for noninvasive hydration sensing,” IEEE journal of microwaves, pp. 1-12, 2022.
[62]S. Bing, K. Chawang, and J.-C. Chiao, “A radio-frequency planar resonant loop for noninvasive monitoring of water content,” in 2022 IEEE Sensors. Dallas, TX, USA: IEEE, Oct. 2022, pp. 1-4.
[63]G. Niu, S. Bing, B. Zhang, and J.-C. Chiao, “Particle filter-based diagnosis and prognosis for human hydration states,” IEEE Sensors Letters, vol. 7, no. 9, pp. 1-4, 2023.
[64]J. Chiao and S. Bing, “Noninvasive water content sensor,” Jan. 11 2024, U.S. patent application Ser. No. 18/342,881.
[65]S. Bing, K. Chawang, and J.-C. Chiao, “A resonant coupler for subcutaneous implant,” Sensors, vol. 21, no. 23, p. 8141, Dec. 6, 2021.
[66]J. Chiao and S. Bing, “Resonant coupler systems and methods for implants,” Dec. 22 2022, U.S. patent application Ser. No. 17/808,033.
[67]S. Bing, K. Chawang, and J. C. Chiao, “A tuned microwave resonant system for subcutaneous imaging,” Sensors, vol. 23, no. 6, p. 3090, Mar. 13, 2023.
[68]S. Bing and J.-C. Chiao, “A planar conformal microwave resonator for subcutaneous imaging,” in 2023 IEEE International Symposium onAntennas and Propagation and USNC-URSI Radio Science Meeting (USNC-URSI), Portland, Oregon, USA, July 23-28, 2023, pp. 335-336.
[69]S. Bing, K. Chawang, and J.-C. Chiao, “A tuned microwave resonant sensor for skin cancerous tumor diagnosis,” IEEE journal of electromagnetics, RF and microwaves in medicine and biology, pp. 1-8, 2023.
[70]S. Bing, K. Chawang, and J. C. Chiao, “Resonant coupler designs for subcutaneous implants,” in 2021 IEEE Wireless Power Transfer Conference (WPTC), Jun. 1, 2021, pp. 1-4.
[71]S. Bing, K. Chawang, and J.-C. Chiao, “A tuned microwave resonator on flexible substrate for nondestructive water content sensing in fruits,” IEEE Journal of Selected Areas in Sensors, vol. 1, pp. 93-104, 2024.
[72]K. Mukai, T. Fujii, Y. Nakajima, A. Ishida, M. Kato, M. Takahashi, M. Tsuda, N. Hashiba, N. Mori, A. Yamanaka, and T. Nakatani, “Factors affecting superficial vein visibility at the upper limb in healthy young adults: A cross-sectional observational study,” The journal of vascular access, vol. 21, no. 6, pp. 900-907, Nov. 1, 2020.
[73]A. Chandrashekar, J. Garry, A. Gasparis, and N. Labropoulos, “Vein wall remodeling in patients with acute deep vein thrombosis and chronic postthrombotic changes,” Journal of thrombosis and haemostasis, vol. 15, no. 10, pp. 1989-1993, Oct. 2017.
[74]N. L. PhD, K. L. S. BS, I. E. S. MD, and J. R. MD, “Saphenousvein wall thickness in age and venous reflux-associated remodeling in adults,” Journal of vascular surgery. Venous and lymphatic disorders (New York, NY), vol. 5, no. 2, pp. 216-223, Mar. 1, 2017.
[75]K. Mukai, Y. Nakajima, T. Nakano, M. Okuhira, A. Kasashima, R. Hayashi, M. Yamashita, T. Urai, and T. Nakatani, “Safety of venipuncture sites at the cubital fossa as assessed by ultrasonography,” Journal of patient safety, vol. 16, no. 1, pp. 98-105, Mar. 2020.
[76]S. A. R. Naqvi, M. Manoufali, B. Mohammed, A. T. Mobashsher, D. Foong, and A. M. Abbosh, “In vivo human skin dielectric properties characterization and statistical analysis at frequencies from 1 to 30 ghz,” IEEE transactions on instrumentation and measurement, vol. 70,pp. 1-10, 2021.
[77]H. N. Mayrovitz, S. Carson, and M. Luis, “Male-female differences in forearm skin tissue dielectric constant,” Clinical physiology and functional imaging, vol. 30, no. 5, pp. 328-332, Sep. 2010.
[78]H. N. Mayrovitz, A. Grammenos, K. Corbitt, and S. Bartos, “Young adult gender differences in forearm skin-to-fat tissue dielectric constant values measured at 300 mhz,” Skin research and technology, vol. 22, no. 1, pp. 81-88, Feb. 2016.
[79]G. Maenhout, T. Markovic, I. Ocket, and B. Nauwelaers, “Effect of open-ended coaxial probe-to-tissue contact pressure on dielectric measurements,” Sensors, vol. 20, no. 7, p. 2060, Apr. 6, 2020.
[80]X. Feng, G.-Y. Li, A. Ramier, A. M. Eltony, and S.-H. Yun, “Invivo stiffness measurement of epidermis, dermis, and hypodermis using broadband rayleigh-wave optical coherence elastography,” Acta biomaterialia, vol. 146, pp. 295-305, Jul. 1, 2022.
[81]A. F. Ghouri, W. Mading, and K. Prabaker, “Accidental intraarterial drug injections via intravascular catheters placed on the dorsum of the hand,” Anesthesia and analgesia, vol. 95, no. 2, pp. 487-491, Aug. 1, 2002.
[82]B. Scheer, A. Perel, and U. J. Pfeiffer, “Clinical review: complications and risk factors of peripheral arterial catheters used for haemodynamic monitoring in anaesthesia and intensive care medicine,” Critical care (London, England), vol. 6, no. 3, pp. 199-204, Jun. 2002.
[83]K. J. Chin and K. Singh, “The superficial ulnar artery—a potential hazard in patients with difficult venous access,” British journal of anaesthesia: BJA, vol. 94, no. 5, pp. 692-693, May 1, 2005.
[84]E. J. Yang, H. S. Ha, Y. H. Kong, and S. J. Kim, “Ultrasound-guided internal jugular vein catheterization in critically ill pediatric patients,” Clinical and experimental pediatrics, vol. 58, no. 4, pp. 136-141, Apr. 1, 2015.
[85]M. Rodriguez-NiedenfUhr, T. Vizquez, L. Nearn, B. Ferreira, I. Parkin, And J. R. Sañudo, “Variations of the arterial pattern in the upper limb revisited: a morphological and statistical study, with a review of the literature,” Journal of anatomy, vol. 199, no. 5, pp. 547-566, Nov. 2001.
[86]P. Lirk, C. Keller, J. Colvin, H. Colvin, J. Rieder, H. Maurer, and B. Moriggl, “Unintentional arterial puncture during cephalic vein cannulation: case report and anatomical study,” British journal of anaesthesia: BJA, vol. 92, no. 5, pp. 740-742, May 1, 2004.
[87]N. L. Moureau, Vessel Health and Preservation: The Right Approach for Vascular Access, 1st ed. Cham: Springer International Publishing, 2019.
[88]L. Gorski, L. Hadaway, M. Hagle, D. Broadhurst, S. Clare, T. Kleidon, B. Meyer, B. Nickel, S. Rowley, E. Sharpe, and M. Alexander, Infusion Therapy Standards of Practice, 8th ed. Infusion Nurses Society, Jan. 1, 2021.
[89]C. Lake and C. L. Beecroft, “Extravasation injuries and accidental intra-arterial injection,” Continuing education in anaesthesia, critical care & pain, vol. 10, no. 4, pp. 109-113, Aug. 1, 2010.
[90]V. Shivappagoudar and B. George, “Unintentional arterial cannulation during cephalic vein cannulation,” Indian journal of anaesthesia, vol. 57, no. 3, pp. 320-322, May 1, 2013.
[91]G. Borrego-Soto, R. Ortiz-López, and A. Rojas-Martinez, “Ionizing radiation-induced dna injury and damage detection in patients with breast cancer,” Genetics and molecular biology, vol. 38, no. 4, pp. 420-432, Dec. 1, 2015.
[92]A. B. Miller, M. E. Sears, L. L. Morgan, D. L. Davis, L. Hardell, M. Oremus, and C. L. Soskolne, “Risks to health and well-being from radio-frequency radiation emitted by cell phones and other wireless devices,” Frontiers in public health, vol. 7, p. 223, Aug. 13, 2019.
[93]“Fcc radio frequency (rf) safety faq.” [Online]. Available: www.fcc.gov/engineering-technology/electromagnetic-compatibility-division/radio-frequency-safety/faq/rf-safety.
[94]I. International Commission on Non-Ionizing Radiation Protection, “Guidelines for limiting exposure to electromagnetic fields (100 khz to 300 ghz),” Health physics, vol. 118, no. 5, pp. 483-524, May 2020.
[95]W. H. Organization, “Who guidelines on drawing blood: best practices in phlebotomy,” 2010. [Online]. Available: www.who.int/iris/handle/10665/44294.
[96]C. Cairns and K. Kang, “National hospital ambulatory medical care survey: 2021 emergency department summary tables,” Tech. Rep., 2021. [Online]. Available: ftp.cdc.gov/pub/HealthStatistics/NCHS/DatasetDocumentation/NHAMCS/doc21-ed-508.pdf.
[97]K. Black, M. Pusic, D. Harmidy, and D. McGillivray, “Pediatric intra- venous insertion in the emergency department: Bevel up or bevel down?” Pediatric emergency care, vol. 21, no. 11, pp. 707-711, Nov. 2005.
[98]O. Y. Buowari, “Complications of venipuncture,” Advances in bioscience and biotechnology, vol. 4, no. 1, pp. 126-128, 2013.
[99]L. Dougherty, “Intravenous therapy in older patients,” Nursing standard, vol. 28, no. 6, pp. 50-58, Oct. 9, 2013.
[100]P. Larsen, D. Eldridge, J. Brinkley, D. Newton, D. Goff, T. Hartzog, N. Saad, and R. Perkin, “Pediatric peripheral intravenous access: Does nursing experience and competence really make a difference?” Journal of infusion nursing, vol. 33, no. 4, pp. 226-235, Jul. 2010.
[101]A. Roggan, M. Friebel, K. Do\ddot{ } rschel, A. Hahn, and G. Mu\ddot{ } ller, “Optical properties of circulating human blood in the wavelength range 400-2500 nm,” Journal of Biomedical Optics, vol. 4, no. 1, pp. 36-46, Jan. 1999.
[102]K. Mukai, Y. Nakajima, T. Nakano, M. Okuhira, A. Kasashima, R. Hayashi, M. Yamashita, T. Urai, and T. Nakatani, “Safety of venipuncture sites at the cubital fossa as assessed by ultrasonography,” Journal of Patient Safety, vol. 16, no. 1, pp. 98-105, Mar. 2020.
[103]V. P. Zharov, S. Ferguson, J. F. Eidt, P. C. Howard, L. M. Fink, and M. Waner, “Infrared imaging of subcutaneous veins,” Lasers in surgery and medicine, vol. 34, no. 1, pp. 56-61, Jan. 2004.
[104]S. J. Curtis, W. R. Craig, E. Logue, B. Vandermeer, A. Hanson, and T. Klassen, “Ultrasound or near-infrared vascular imaging to guide peripheral intravenous catheterization in children: a pragmatic randomized controlled trial,” Canadian Medical Association journal (CMAJ), vol. 187, no. 8, pp. 563-570, May 19, 2015.
[105]J.-C. Chiao, C. Li, J. Lin, R. H. Caverly, J. C. M. Hwang, H. Rosen, and A. Rosen, “Applications of microwaves in medicine,” IEEE journal of microwaves, vol. 3, no. 1, pp. 134-169, Jan. 2023.
[106]J.-C. Chiao, S. Bing, and K. Chawang, “Review on noninvasive radio- frequency sensing for closed-loop body health management,” in 2021 IEEE International Symposium on Radio-Frequency Integration Technology (RFIT). Piscataway: IEEE, Aug. 25, 2021, pp. 1-3.
[107]M. Baghelani, Z. Abbasi, M. Daneshmand, and P. E. Light, “Non- invasive continuous-time glucose monitoring system using a chipless printable sensor based on split ring microwave resonators,” Scientific reports, vol. 10, no. 1, p. 12980, Jul. 31, 2020.
[108]J. Kilpija\ddot{ } rvi, J. Tolvanen, J. Juuti, N. Halonen, and J. Hannu, “A non- invasive method for hydration status measurement with a microwave sensor using skin phantoms,” IEEE Sensors Journal, vol. 20, no. 2, pp. 1095-1104, Jan. 15, 2020.
[109]S. Bing, K. Chawang, and J. C. Chiao, “A self-tuned method for impedance-matching of planar-loop resonators in conformable wearables,” Electronics, vol. 11, no. 17, p. 2784, Sep. 4 2022.
[110]S. Bing, K. Chawang, and J.-C. Chiao, “A flexible tuned radio-frequency planar resonant loop for noninvasive hydration sensing,” IEEE journal of microwaves, pp. 1-12, 2022.
[111]G. Niu, S. Bing, B. Zhang, and J.-C. Chiao, “Particle filter-based diagnosis and prognosis for human hydration states,” IEEE Sensors Letters, vol. 7, no. 9, pp. 1-4, 2023.
[112]S. Bing, K. Chawang, and J.-C. Chiao, “A radio-frequency planar resonant loop for noninvasive monitoring of water content,” in 2022 IEEE Sensors. Dallas, TX, USA: IEEE, Oct. 2022, pp. 1-4.
[113]S. Bing, K. Chawang, and J. Chiao, “A resonant coupler for subcutaneous implant,” Sensors, vol. 21, no. 23, p. 8141, Dec. 6, 2021.
[114]S. Bing, K. Chawang, and J.-C. Chiao, “A tuned microwave resonant system for subcutaneous imaging,” Sensors, vol. 23, no. 6, p. 3090, Mar. 13, 2023.
[115]S. Bing, K. Chawang, and J. C. Chiao, “A tuned microwave resonant sensor for skin cancerous tumor diagnosis,” IEEE journal of electro-magnetics, RF and microwaves in medicine and biology, pp. 1-8, 2023.
[116]S. Bing, K. Chawang, and J.-C. Chiao, “Resonant coupler designs for subcutaneous implants,” in 2021 IEEE Wireless Power Transfer Conference (WPTC), Jun. 1, 2021, pp. 1-4.
[117]S. Bing, K. Chawang, and J. C. Chiao, “A tuned microwave resonator on flexible substrate for nondestructive water content sensing in fruits,” IEEE Journal of Selected Areas in Sensors, vol. 1, pp. 93-104, 2024.
[118]A. Chandrashekar, J. Garry, A. Gasparis, and N. Labropoulos, “Vein wall remodeling in patients with acute deep vein thrombosis and chronic post- thrombotic changes,” Journal of thrombosis and haemostasis, vol. 15, no. 10, pp. 1989-1993, Oct. 2017.
[119]N. Labropoulos, K. Summers, I. Escotto, and J. Raffetto, “Saphenous vein wall thickness in age and venous reflux-associated remodeling in adults,” Journal of vascular surgery. Venous and lymphatic disorders (New York, NY), vol. 5, no. 2, pp. 216-223, Mar. 1, 2017.
[120]D. Andreuccetti, R. Fossi, and C. Petrucci, “An internet resource for the calculation of the dielectric properties of body tissues in the frequency range 10 Hz-100 GHz,” niremf.ifac.cnr.it/tissprop/, 1997, iFAC- CNR, Florence (Italy), Based on data published by C.Gabriel et al.in 1996.
[121]K. Mukai, T. Fujii, Y. Nakajima, A. Ishida, M. Kato, M. Takahashi, M. Tsuda, N. Hashiba, N. Mori, A. Yamanaka, and T. Nakatani, “Factors affecting superficial vein visibility at the upper limb in healthy young adults: A cross-sectional observational study,” The journal of vascular access, vol. 21, no. 6, pp. 900-907, Nov. 1, 2020.
[122]S. Bing, K. Chawang, and J.-C. Chiao, “A noninvasive vein finder based on a tuned microwave loop resonator,” IEEE Journal of Microwaves, 2024.

The references listed herein are incorporated herein in their entirety by reference.

What is claimed is:

1. A system for detecting a blood vessel, comprising:
a resonator mounted on a substrate; and
a processor connected to the resonator;
wherein the resonator and the processor are configured to detect at least one of resonant frequencies and reflection coefficient magnitudes when the resonator is applied to a skin surface of a subject.

2. The system of claim 1, wherein the resonator is a tuned loop resonator comprising an outer loop and a metal pad disposed within the outer loop and concentric with the outer loop such that a gap width between the outer loop and the metal pad is constant.

3. The system of claim 2, wherein the metal pad has a hole at a center thereof, the hole configured to receive an injection needle or visualization of the skin.

4. The system of claim 1, wherein the processor is a network analyzer, a vector network analyzer, or a spectrum analyzer.

5. The system of claim 1, wherein at least one of:
the resonator and the processor are configured to detect differences in at least one of the resonant frequencies and the reflection coefficient magnitudes when the resonator is applied to more than one location on the skin surface;
the resonator and the processor are configured to detect differences in at least one of the resonant frequencies and the reflection coefficient magnitudes when the resonator is applied to the skin surface over the blood vessel and when the resonator is applied to the skin surface over tissue surrounding the blood vessel;
the resonator and the processor are configured to detect changes in at least one of the resonant frequencies and the reflection coefficient magnitudes over time when the resonator is applied to the skin surface;
the resonator is applied to the skin surface over a blood vessel; or
the resonator is selected from RF, microwave, millimeter-wave, and terahertz-frequency sensors; infrared, near-infrared, and ultraviolet optical sensors; magnetic sensors; acoustic sensors; and combination thereof.

6. The system of claim 1, wherein the system further comprises one or more additional resonators configurable in an array on the skin surface.

7. A kit for a system for detecting blood vessels, comprising:
a resonator mounted on a substrate; and
a processor connected to the resonator;
wherein the resonator and the processor are configured to detect at least one of resonant frequencies and reflection coefficient magnitudes when the resonator is applied to a skin surface of a subject; and
one or more tools for manipulation of the resonator and instructions for use of the kit.

8. The kit of claim 7, wherein the resonator is a tuned loop resonator comprising an outer loop and a metal pad disposed within the outer loop and concentric with the outer loop such that a gap width between the outer loop and the metal pad is constant.

9. The kit of claim 8, wherein the metal pad has a hole at a center thereof, the hole configured to receive an injection needle or visualization of the skin.

10. The kit of claim 7, wherein the processor is a network analyzer, a vector network analyzer, or a spectrum analyzer.

11. The kit of claim 7, wherein at least one of:
the resonator and the processor are configured to detect differences in at least one of the resonant frequencies and the reflection coefficient magnitudes when the resonator is applied to more than one location on the skin surface;
the resonator and the processor are configured to detect differences in at least one of the resonant frequencies and the reflection coefficient magnitudes when the resonator is applied to the skin surface over the blood vessel and when the resonator is applied to the skin surface over tissue surrounding the blood vessel;
the resonator and the processor are configured to detect changes in at least one of the resonant frequencies and the reflection coefficient magnitudes over time when the resonator is applied to the skin surface;
the resonator is applied to the skin surface over a blood vessel; or
the resonator is selected from RF, microwave, millimeter-wave, and terahertz-frequency sensors; infrared, near-infrared, and ultraviolet optical sensors; magnetic sensors; acoustic sensors; and combination thereof.

12. The kit of claim 7, wherein the system further comprises one or more additional resonators configurable in an array on the skin surface.

13. A method of detecting a blood vessel, comprising:
providing a subject in need of detection of the blood vessel;
providing a system for detecting the blood vessel comprising:
a resonator mounted on a substrate; and
a processor connected to the resonator;
wherein the resonator and the processor are configured to detect at least one of resonant frequencies and reflection coefficient magnitudes when the resonator is applied to a skin surface of the subject;
applying the resonator to a plurality of locations on a skin surface of the subject;
detecting the at least one of the resonant frequencies and the reflection coefficient magnitudes at each of the plurality of locations on the skin surface; and
identifying the blood vessel from differences among the at least one of the resonant frequencies and the reflection coefficient magnitudes detected.

14. The method of claim 13, wherein the resonator is a tuned loop resonator comprising an outer loop and a metal pad disposed within the outer loop and concentric with the outer loop such that a gap width between the outer loop and the metal pad is constant.

15. The method of claim 13, wherein the metal pad has a hole at a center thereof, the hole configured to receive an injection needle or visualization of the skin.

16. The method of claim 13, wherein the processor uses a network analyzer, a vector network analyzer, or a spectrum analyzer.

17. The method of claim 13, wherein at least one of
the resonator and the processor are configured to detect differences in the at least one of the resonant frequencies and the reflection coefficient magnitudes when the resonator is applied to more than one location on the skin surface;
the resonator and the processor are configured to detect differences in the at least one of the resonant frequencies and the reflection coefficient magnitudes when the resonator is applied to the skin surface over the blood vessel and when the resonator is applied to the skin surface over tissue surrounding the blood vessel;
the resonator and the processor are configured to detect changes in the at least one of the resonant frequencies and the reflection coefficient magnitudes over time when the resonator is applied to the skin surface;
the resonator is applied to the skin surface over a blood vessel; or
the resonator is selected from RF, microwave, millimeter-wave, and terahertz-frequency sensors; infrared, near-infrared, and ultraviolet optical sensors; magnetic sensors; acoustic sensors; and combination thereof.

18. The method of claim 13, wherein the system further comprises one or more additional resonators configurable in an array on the skin surface.

19. A processor-implemented method for detecting a blood vessel and blood flow in the blood vessel using a machine learning model, wherein the method comprises:
performing, using a processor, a scan of a tissue with a resonator sensor to conduct one or more raster scans that generate pixels of data to create an image;
wherein the resonator sensor detects a reflection coefficient s₁₁ within a range of frequencies, wherein a magnitude and phase of s₁₁ as a function of frequency are recorded; and
determining a resonant frequency and magnitude at a location on skin to generate a first and a second images,
wherein a first image contains pixels of the resonant frequencies and the second image contains pixels of the |s₁₁|, wherein the resonant-frequency image shows a location of a vein as a line of pixels wherein the resonant frequencies of the vein under the skin are lower than neighbor pixels.

20. The processor-implemented method of claim 19, wherein a frequency with a minimum |s₁₁| is treated as a resonant frequency.

21. The processor-implemented method of claim 19, further comprising combining a data set of the first and second images to generate a resonant-frequency map of the vein location by:
normalizing a datasets of the first and second images to a minimum and maximum range, wherein the normalization ranges between 0 and 1, with 0 being a lowest resonant frequency and 1 being a highest resonant frequency in the resonant-frequency map, and 0 being the lowest |s₁₁| and 1 being the highest |s₁₁| in the |s₁₁|map, respectively;
generating a first and a second map with a scale from 0 to 1, wherein a weighting value w is used to emphasize a specific map, wherein the value w is between 0.01 and 0.99; and
wherein the first map is given a value of x while the second map is given 1-w;
generating an integrated map with each pixel containing a sum of a pixel value times w from the first map and the pixel value times (1-w) from the second map;
wherein, if the resonant-frequency map has a line of pixels with values lower than others, the resonant-frequency map is given a larger value w; or
wherein, if the |s₁₁| map has a line of pixels with values lower than others, the |s₁₁|map is given a larger value w, while the resonant-frequency map is given a smaller value of (1-w);
generating a series of integrated maps comprising a series of varying w values;
comparing a contrasts of the first and second maps, wherein a highest contrast map gives the vein location, as a line, and determines an optimal w value; and
using an |s₁₁| image to find a vein location.

22. The processor-implemented method of claim 19, further comprising obtaining an ultrasound image of the tissue with an ultrasound transducer array.

23. The processor-implemented method of claim 22, wherein the resonator sensor identifies the vein along a medial/lateral and proximal/distal location, and concurrently an ultrasound cross-sectional image indicates the vein depth under the skin.

24. The processor-implemented method of claim 22, wherein a machine learning model increases a sensitivity of the vein localization by taking two or more scans of the same location of a tissue wherein two or more pixel maps are collected for known vein locations, segmenting the two or more pixel maps into two or more smaller pixel maps for training; rotating the two or more smaller pixel maps to change vein locations; determining a ground truth for each of the two or more smaller pixel maps that contain vein locations in certain pixels or without vein pixels.

25. The processor-implemented method of claim 22, further comprising using the machine learning model on new scans to generate vein locations maps at one or more different depths, wherein optionally a depth of the vein can be verified by an ultrasound transducer.

26. One or more non-transitory computer-readable storage mediums storing one or sequences of instructions, which when executed by one or more processors, causes a method for detecting a blood vessel and blood flow in the blood vessel using a machine learning model, wherein the method comprises:
performing, using a processor, a scan of a tissue with a resonator sensor to conduct one or more raster scans that generate pixels of data to create an image;
wherein the resonator sensor detects a reflection coefficient s₁₁ within a range of frequencies, wherein a magnitude and phase of s₁₁ as a function of frequency are recorded; and
determining a resonant frequency and a magnitude at a location on the skin to generate a first and a second images,
wherein a first image contains pixels of the resonant frequencies and the second image contains pixels of the |s₁₁|, wherein the resonant-frequency image shows a location of a vein as a line of pixels wherein the resonant frequencies of the vein under the skin are lower than neighbor pixels.

27. The one or more non-transitory computer-readable storage mediums storing one or sequences of instructions of claim 26, wherein a frequency with a minimum |s₁₁| is treated as a resonant frequency.

28. The one or more non-transitory computer-readable storage mediums storing one or sequences of instructions of claim 26, further comprising combining a data set of the first and second images to generate a resonant-frequency map of the vein location by:
normalizing a datasets of the first and second images to a minimum and maximum range, wherein the normalization ranges between 0 and 1, with 0 being a lowest resonant frequency and 1 being a highest resonant frequency in the resonant-frequency map, and 0 being the lowest |s₁₁| and 1 being the highest |s₁₁| in the |s₁₁|map, respectively;
generating a first and a second map with a scale from 0 to 1, wherein a weighting value w is used to emphasize a specific map, wherein the value w is between 0.01 and 0.99; and
wherein the first map is given a value of x while the second map is given 1-w;
generating an integrated map with each pixel containing a sum of a pixel value times w from the first map and the pixel value times (1-w) from the second map;
wherein, if the resonant-frequency map has a line of pixels with values lower than others, the resonant-frequency map is given a larger value w; or
wherein, if the |s₁₁| map has a line of pixels with values lower than others, the |s₁₁|map is given a larger value w, while the resonant-frequency map is given a smaller value of (1-w);
generating a series of integrated maps comprising a series of varying w values;
comparing a contrasts of the first and second maps, wherein a highest contrast map gives the vein location, as a line, and determines an optimal w value; and
using an |s₁₁| image to find a vein location.

29. The one or more non-transitory computer-readable storage mediums storing one or sequences of instructions of claim 26, further comprising obtaining an ultrasound image of the tissue with an ultrasound transducer array.

30. The one or more non-transitory computer-readable storage mediums storing one or sequences of instructions of claim 29, wherein the resonator sensor identifies the vein along a medial/lateral and proximal/distal location, and concurrently an ultrasound cross-sectional image indicates the vein depth under the skin.

31. The one or more non-transitory computer-readable storage mediums storing one or sequences of instructions of claim 26, wherein a machine learning model increases a sensitivity of the vein localization by taking two or more scans of the same location of a tissue wherein two or more pixel maps are collected for known vein locations, segmenting the two or more pixel maps into two or more smaller pixel maps for training; rotating the two or more smaller pixel maps to change vein locations; determining a ground truth for each of the two or more smaller pixel maps that contain vein locations in certain pixels or without vein pixels.

32. The one or more non-transitory computer-readable storage mediums storing one or sequences of instructions of claim 31, further comprising using the machine learning model on new scans to generate vein locations maps at one or more different depths, wherein optionally a depth of the vein can be verified by an ultrasound transducer.
